(19) United States
(12) Patent Application Publication
(10) Pub. No.: US 2022/0047651 A1
(43) Pub. Date: Feb. 17, 2022

(54) READY-TO-USE PROBIOTIC COMPOSITIONS AND USES THEREOF

(71) Applicants: Edward Robb, Parkville, MO (US); Richard Strafehl, Toronto (CA); Healthy Cow Corporation, Toronto (CA)

(72) Inventors: Edward Robb, Parkville, MO (US); Richard Strafehl, Toronto (CA)

(73) Assignee: Healthy Cow Corporation, Toronto, ON (CA)

(21) Appl. No.: 17/416,055

(22) PCT Filed: Dec. 18, 2019
(86) PCT No.: PCT/US19/67226

§ 371 Date: Jun. 18, 2021

Related U.S. Application Data
(60) Provisional application No. 62/782,066, filed on Dec. 19, 2018.

(51) Int. Cl. A61K 35/744; A61K 47/36; A61M 31/00; A61K 35/747; A61P 31/00; A61P 15/02

(52) U.S. Cl. CPC A61K 35/744; A61K 47/36; A61M 31/00; A61M 2210/1475; A61P 31/00; A61P 15/02; A61K 35/747

(57) ABSTRACT

Probiotic compositions suitable for intravaginal administration to a non-human animal comprising one or more strains of bacteria native to the vagina of the species of animal and a non-aqueous base, probiotic products, kits, and systems comprising the probiotic compositions, and methods of using the probiotic compositions, products, kits, and systems.

### 1. CROSS REFERENCE TO RELATED APPLICATIONS

This application claims priority benefit of U.S. provisional application No. 62/782,066, filed Dec. 19, 2018, the contents of which are incorporated herein in their entireties by reference thereto.

### 2. BACKGROUND

Probiotics have demonstrated utility in health and disease in both man and animals. Cattle are very prone to developing uterine and systemic infections post calving and these infections are commonly treated with systemic antibiotics. Agricultural use of antibiotics is associated with contributing to overall antimicrobial resistance in both man and animals and these drugs are also sources of contamination to meat, milk and the environment. In cattle, the majority of uterine infections (e.g., metritis) are caused by local environmental organisms that contaminate the female reproductive tract during late pregnancy and post calving. Common pathogens found in the female reproductive tract include Escherichia coli, Truperella pyogenes, and Fusobacterium necrophorum.

Clinical metritis in cows is characterized by fever, a foul-fetid vulvar discharge, a uterus with excess fluid and lacking tone, and a cow that appears depressed and off-feed. Clinical metritis is most commonly seen in the first 10 days post-calving. Cows that have clinical metritis exhibit poor reproductive performance with irregular estrous cycles, lower conception rates and greater intervals from calving to pregnancy. Infection of the uterus with E. coli appears to pave the way for subsequent infection with other bacteria such as T. pyogenes and Gram-negative anaerobes. Clostridium species of bacteria may be part of the bacterial mix that results in the most acute and severe clinical metritis.

The cow's immune system is normally suppressed at the time of calving. The normal suppression can be further aggravated by ketosis, milk fever, and trace mineral and vitamin deficiencies. In addition, difficult calving, stillbirths, twins and retained fetal membranes put the cow at a greater risk for the development of metritis. The current treatment of choice is a 5-day regimen of an antibiotic labeled for the treatment of metritis and appropriate supportive therapy. Primary measures for prevention of metritis include appropriate supplementation of trace minerals and vitamins, feeding a diet with appropriate levels of calcium and a negative dietary-cation difference to prevent milk fever, minimization of negative energy balance around calving time by managing pen moves and preventing over-crowding, feeding appropriate transition rations, maintaining a clean, dry maternity environment, and providing well managed assistance when a calving difficulty occurs.

The anatomy of the cow and current husbandry practices makes it hard to reestablish and/or supplement natural vaginal microflora. The application of active growing Lactobacillus cultures in liquid, water based media has been explored (see, e.g., Deng et al., 2015, PLoS One. 10(4):e0124167; Ametaj et al., 2014, Research in Veterinary Science 96: 365-370), but use of active growing Lactobacillus cultures in liquid, water based media is impractical for routine and cost effective use in commercial farming operations because liquid cultures are not storage stable and require reconstitution prior to use.

Thus, new approaches for reestablishing and/or supplementing natural vaginal microflora in cows and other non-human animals are needed.

### 3. SUMMARY

The present disclosure provides probiotic compositions suitable for intravaginal administration to a non-human animal (e.g., a ruminant such as a dairy cow, or other domesticated animal), products, kits, and systems comprising a probiotic composition, and methods of using the compositions, products, kits, and systems. The probiotic compositions of the disclosure are useful, for example, for repopulating and/or maintaining a healthy vaginal microbiome in an animal (e.g., in dairy cows) before and/or after labor.

In one aspect, the disclosure provides probiotic compositions in gel form suitable for intravaginal administration to an animal. The probiotic compositions can comprise one or more strains of probiotic bacteria and a non-aqueous base (e.g., an oil-based base). The probiotic compositions can further comprise one or more prebiotics, such one or more dried fermentation products, that can help the probiotic bacteria to grow, expand, and colonize the vaginal tract after administration. Without being bound by theory, it is believed that a fermentation product, which can contain nutrients and cofactors that the bacteria would have been exposed to during the fermentation used to produce the bacteria, can serve as a “wake up catalyst” once the probiotic composition is hydrated in vivo, thus decreasing the amount of time required for the probiotic bacteria to grow, expand, and colonize the vaginal tract. Exemplary probiotic compositions of the disclosure are described in Section 5.2 and numbered embodiments 1 to 284, infra.

The probiotic compositions of the disclosure have several advantages over water-based probiotic compositions. For example, probiotic compositions of the disclosure can “stick” to the vaginal wall better than water-based compositions, helping to prevent the probiotic bacteria from being expelled from the vagina before the bacteria have a chance to “wake up” and populate the vaginal tract. The probiotic compositions of the disclosure can also be transported and stored in a ready-to-use format, and with a greatly prolonged shelf-life as compared to probiotic compositions formulated with a water-based carrier such as skim milk (e.g., as described in Deng et al., 2015, PLoS One. 10(4):e0124167). Thus, the use of a non-aqueous base allows for ready-to-use probiotic products, avoiding the need to reconstitute the probiotic composition in a water-based carrier prior to use.

The disclosure further provides probiotic products comprising a probiotic composition of the disclosure. A probiotic product can comprise, for example, a probiotic composition packaged within a container or capsule or a probiotic composition in the form of a suppository. The probiotic product is preferably a ready-to-use product comprising an amount of a probiotic composition in a single or multi-dose container, such as a cartridge or syringe. Exemplary probiotic products of the disclosure are described in Section 5.3.1 and numbered embodiments 285 to 321, infra.

The disclosure further provides kits comprising a probiotic product of the disclosure. The kits of the disclosure can comprise an applicator tube that can be attached to the container of a probiotic product and/or can comprise an applicator gun (for example, when the container is a cartridge). Exemplary kits of the disclosure are described in Section 5.3.2 and numbered embodiments 322 to 386, infra.

The disclosure further provides systems comprising a kit of the disclosure configured for use. Exemplary systems are described in Section 5.3.3 and numbered embodiments 387 to 389, infra.

The disclosure further provides methods of using the probiotic compositions, probiotic products, kits, and systems of the disclosure.

In one aspect, the disclosure provides methods of introducing one or more strains of bacteria to the vagina of a non-human animal comprising administering an amount of a probiotic composition of the disclosure to the vagina of the animal.

In another aspect, the disclosure provides methods of treating a uterine infection (e.g., metritis, endometritis, or pyometra) or lowering the risk of contracting a uterine infection in a non-human animal comprising administering an amount of a probiotic composition of the disclosure to the vagina of the animal.

In another aspect, the disclosure provides methods of treating a urogenital infection (e.g., a urinary tract infection or vaginitis) or lowering the risk of contracting a urogenital infection in a non-human animal comprising administering an amount of a probiotic composition of the disclosure to the vagina of the animal.

In another aspect, the disclosure provides methods of promoting the establishment or maintenance of a heathy vaginal microbiome in a non-human animal comprising administering an amount of a probiotic composition of the disclosure to the vagina of the animal.

In another aspect, the disclosure provides methods of accelerating resumption of ovarian cyclicity in a non-human animal following labor comprising administering an amount of a probiotic composition of the disclosure to the vagina of the animal.

In another aspect, the disclosure provides methods of reducing the number of days open in a non-human animal (e.g., a dairy cow) following labor comprising administering an amount of a probiotic composition of the disclosure to the vagina of the animal.

In another aspect, the disclosure provides methods of reducing the incidence of retained placenta in a non-human animal (e.g., a dairy cow) following labor comprising administering an amount of a probiotic composition of the disclosure to the vagina of the animal.

In another aspect, the disclosure provides methods of increasing the amount of colostrum and/or increasing the immunoglobulin content of colostrum produced by a non-human animal (e.g., a dairy cow), comprising administering an amount of a probiotic composition of the disclosure to the vagina of a pregnant non-human animal prior to labor.

The probiotic composition administered in the methods of the disclosure can be, in various embodiments, a probiotic composition which is part of a probiotic product, kit, or system of the disclosure. Exemplary methods of using the probiotic compositions, products, kits, and systems of the disclosure are described in Section 5.4 and numbered embodiments 390 to 520, infra.

### 4. BRIEF DESCRIPTION OF THE FIGURES

FIG. 1 shows an exemplary system of the disclosure. The system shown in FIG. 1 comprises an exemplary applicator tube attached to a cartridge containing a probiotic composition, with the cartridge positioned within an applicator gun.

### 5. DETAILED DESCRIPTION

The present disclosure provides probiotic compositions suitable for intravaginal administration to a non-human animal, products, kits, and systems comprising a probiotic composition, and methods of using the compositions, products, kits, and systems. The non-human animal can be, for example, a non-human animal such as a domestic animal. Domestic animals for which probiotic compositions can be made include ruminants, such as cows (e.g., dairy cows), sheep, and goats, horses, pigs, dogs, and cats. Cows can be of the species Bos taurus (e.g., a cow of the Holstein, Brown Swiss, Guernsey, Ayrshire, Jersey, Red and White, or Milking Shorthorn breeds, or a mixed breed of any of the foregoing), which is the prevalent dairy cow species in the United States, Canada, Europe, Australia, and New Zealand, or another species such as Bos indicus (e.g., a cow of the Sahiwal or Gir breeds), which is a species often used for dairy production in warmer climates, for example in the Indian subcontinent, Africa, and Brazil.

Exemplary probiotic compositions of the disclosure are described in Section 5.2 and numbered embodiments 1 to 284, infra. Exemplary probiotic products of the disclosure are described in Section 5.3.1 and numbered embodiments 285 to 321, infra. Exemplary kits of the disclosure are described in Section 5.3.2 and numbered embodiments 322 to 386, infra. Exemplary systems of the disclosure are described in Section 5.3.3 and numbered embodiments 387 to 389, infra. Exemplary methods of using the probiotic compositions, products, kits, and systems of the disclosure are described in Section 5.4 and numbered embodiments 390 to 520, infra.

### 5.1. Definitions

Unless defined otherwise herein, all technical and scientific terms used herein have the same meaning as commonly understood by one of ordinary skill in the art. Various scientific dictionaries that include the terms included herein are well known and available to those in the art.

As used herein, the singular forms “a”, “an” and “the” include plural referents unless the content and context clearly dictates otherwise. Thus, for example, reference to “an oil” includes a combination of two oils, a combination of three oils, and the like.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

The term “about” is used throughout the specification in front of a number to show that the number is not necessarily exact (e.g., to account for variations in measurement accuracy and/or precision, timing, etc.). It should be understood that a disclosure of “about X,” where X is a number is also a disclosure of “X.” Thus, for example, a disclosure of an embodiment in which administrations of a probiotic composition are separated by “about 1 week” is also a disclosure of an embodiment in which administrations of the probiotic composition are separated by “1 week.”

As used herein, the terms “comprising,” “comprises,” and the like encompass the more restrictive terms “consisting essentially of,” “consists essentially of,” “consisting of,” “consists of,” and the like. It should be understood that disclosure of an embodiment using the terms “comprising,” “comprises” or the like is also a disclosure of embodiments with the more restrictive terms “consisting essentially of,” “consists essentially of,” “consisting of,” “consists of,” or the like, whether or not the more restrictive terms are explicitly recited. Thus, for example, disclosure of an embodiment of a probiotic composition in which the bacteria “comprise” lactic acid bacteria is also a disclosure of an embodiment in which the bacteria “consist essentially of” lactic acid bacteria and a disclosure of an embodiment in which the bacteria “consist of” lactic acid bacteria.

When the terms “animal” and “healthy animals” are referred to in an embodiment or claim, it should be understood that the “animal” and the “healthy animals” are of the same species.

As used herein, unless required otherwise by context, a “cow” refers to a female animal of the Bos genus (e.g., Bos taurus or Bos indicus) without regard to whether the animal has never had a calf (commonly referred to as a “heifer”) or has had a calf. Thus, the term “cow” includes both heifers and animals that have had a calf, unless required otherwise by context.

As used herein, the term “fermentation product” refers to a composition comprising a fermentation broth that has been dried following removal of the biomass. A fermentation product can be produced, for example, by removing biomass (e.g., by centrifugation) from a fermentation broth to produce a depleted fermentation broth and subsequently drying the depleted fermentation broth (e.g., by spray-drying) to produce the fermentation product. Fermentation products can in some embodiments include one or more additional reagents (e.g., a thickener) that were combined with the depleted fermentation broth prior to drying. Because methods for removing biomass from a fermentation broth and methods for drying a may not remove 100% of the biomass and 100% of the water, a fermentation product can contain small amounts of water (e.g., less than 5% by weight) and/or small amounts of biomass (e.g., less than 5% by weight).

As used herein, the terms “labor” and “calving” refer to the process of giving birth to offspring. Unless required otherwise by context, the term “calving” is not limited to labor by a cow, but encompasses labor by other non-human animals.

As used herein, the term “native” in reference to a strain of bacteria native to a species means that the strain of bacteria is naturally found in the vaginas of healthy members of the species. A strain does not necessarily need to be present in all members of the species to be considered a native strain. Thus, for example, a strain of bacteria can be considered native to a species when it is naturally found in one or more populations of healthy animals even though the strain may not be found in all populations of healthy animals.

As used herein, the term “non-aqueous base” means a carrier for one or more strains of bacteria that is not water-based. Preferably, non-aqueous bases and the components thereof are free of water, although the presence of water as a minor component in a non-aqueous base are permitted. In some embodiments, a probiotic composition of the disclosure has less than 5% water by weight of the probiotic composition.

As used herein, the term “prebiotic” refers to a compound or composition of matter (e.g., a fermentation product) that promotes the growth or activity of beneficial microorganisms such as beneficial bacteria.

As used herein, the term “step down” in reference to an applicator tube refers to an abrupt decrease from a first inner diameter of the applicator tube to a second, smaller inner diameter. A step down exists, for example, where a first section an applicator tube having a constant inner diameter is joined to a second section of the applicator tube having a constant inner diameter that is smaller than the inner diameter of the first section.

As used herein, the term “thickener” refers to compound or composition of matter (e.g., a mixture of compounds) that can increase the viscosity of a liquid (e.g., an oil) when added to the liquid. A thickener can function as a gelling agent, forming a gel with the other components of the probiotic composition.

### 5.2. Probiotic Compositions

The disclosure provides probiotic compositions comprising one or more strains of bacteria and a non-aqueous base (e.g., an oil and/or wax based base). Exemplary bacteria and exemplary features of bacteria that can be included in the probiotic compositions of the disclosure are described in Section 5.2.1, infra. Exemplary non-aqueous bases and components that can be included in non-aqueous bases are described in Section 5.2.2, infra.

The probiotic compositions of the disclosure preferably contain less than 5% water (e.g., 4% or less, 3% or less, 2% or less, or 1% or less) as measured by NMR spectroscopy. Other techniques for measuring water content can also be used, for example, neutron scattering, Raman spectroscopy, infrared spectroscopy, differential scanning calorimetry, thermal activity monitor, gravimetric sorption analysis, and thermogravimetric/mass spectrometry. Capacitive sensors that can measure residual humidity in a near vacuum of a freeze-drier can also be used for determining water content of a probiotic composition.

Probiotic compositions of the disclosure comprising one or more liquid components (e.g., one or more oils) and one or more non-liquid components (e.g., crystals, powders, etc.) can be made, for example, by a process comprising combining the non-liquid components, mixing the non-liquid components, and then combining the mixture of non-liquid components with the liquid components (e.g., the liquid components can be added to the mixture of non-liquid components while mixing). For compositions comprising more than one liquid (e.g., more than one oil), the liquids can be combined with each other prior to being combined with the non-liquid components. Alternatively, one or more of the liquids can be combined with the non-liquid components and, subsequently, one or more additional liquids can be added.

Probiotic compositions of the disclosure can also be made by, for example, by a process comprising combining the one or more strains of bacteria (preferably dried, for example by lyophilization or spray-drying) with a pre-formed non-aqueous base. For example, a dried powder comprising the bacteria can be blended with a pre-formed non-aqueous base comprising one or more components as described in Section 5.2.2. Alternatively, a probiotic composition of the disclosure can be made by a process comprising combining the one or more strains of dried bacteria with one or more components of a multi-component non-aqueous base (e.g., an oil and a thickener) to form an intermediate composition, and then combining the intermediate composition with one or more additional components of the non-aqueous base (e.g., a prebiotic).

Thus, it should be understood that the probiotic compositions of the disclosure can be made by various processes, and unless required otherwise by context, the probiotic compositions described herein are not limited to a specific preparation process. It should also be understood that all components of a probiotic composition described herein, other than the one or more strains of bacteria, can be considered a component of the non-aqueous base.

The amount of bacteria included in a probiotic composition (represented as colony forming units (CFU)) can vary, and can be selected so as to provide a desired CFU count in a given volume or mass of the probiotic composition. In some embodiments, the one or more strains of bacteria together comprise 10³ to 10¹⁰ total colony forming units (CFU) per 1 ml of the probiotic composition, e.g., 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰, CFU per 1 ml.

In some embodiments, a probiotic composition of the disclosure comprises 0.2 billion to 0.8 billion, 0.2 billion to 0.6 billion, 0.4 billion to 1 billion, 0.4 billion to 0.8 billion, 0.4 billion to 0.6 billion, 0.6 billion to 1 billion, 0.6 billion to 0.8 billion, 0.8 billion to 1 billion CFU per 1 ml. Compositions having a relatively high CFU count can be made, for example, so that a relatively small amount of the composition needs to be administered to an animal in order to provide a desired total amount of bacteria. Conversely, compositions having a relatively low CFU count can be made, for example, if it is desirous to administer a relatively large amount of the composition (e.g., to distribute the bacteria over a larger area and/or length of the vaginal tract). CFU counts can be determined, for example, by using a standard plate count method. See, e.g., US Food and Drug Administration's Bacteriological Analytical Manual, Edition 8, Revision A, 1998, Chapter 3: Aerobic Plate Count.

The bacteria included in a probiotic composition of the disclosure are preferably dried prior to being incorporated into the probiotic composition. Suitable processes for drying bacteria are known in the art and include spray-drying and lyophilization. Drying the bacteria prior to incorporation into the probiotic composition can help to preserve the viability of the bacteria, thereby extending the shelf-life of the probiotic composition. In some embodiments, a probiotic composition of the disclosure maintains at least 60% of its CFU after 3 months of storage at 20° C. (e.g., 60% to 95%, 60% to 90%, 60% to 80%, or 60% to 70%). In other embodiments, a probiotic composition of the disclosure maintains at least 60% of its CFU after 6 months of storage at 20° C. (e.g., 60% to 95%, 60% to 90%, 60% to 80%, or 60% to 70%). In other embodiments, a probiotic composition of the disclosure maintains at least 60% of its CFU after 9 months of storage at 20° C. (e.g., 60% to 95%, 60% to 90%, 60% to 80%, or 60% to 70%). In other embodiments, a probiotic composition of the disclosure maintains at least 60% of its CFU after 12 months of storage at 20° C. (e.g., 60% to 95%, 60% to 90%, 60% to 80%, or 60% to 70%). In other embodiments, a probiotic composition of the disclosure maintains at least 60% of its CFU after 24 months of storage at 20° C. (e.g., 60% to 95%, 60% to 90%, 60% to 80%, or 60% to 70%).

The consistency of a probiotic composition of the disclosure is preferably that of a gel. Typically, a probiotic composition in the form of a gel flows less freely than water at 25° C. and can have any one of a range of consistencies. For example, a probiotic composition in gel form can be relatively soft (e.g., having a consistency similar to brown mustard), while a different probiotic composition in gel form can be relatively hard (e.g., having a consistency similar to cheddar cheese).

Test methods for measuring consistency have been developed in the lubricant field, and such methods can be used to characterize probiotic compositions of the disclosure. For example, the National Lubricating Grease Institute (NLGI) has developed a numerical grading system to classify lubricants from grade 000 (fluid) to 6 (very hard). In some embodiments, a probiotic composition can have an NLGI consistency grade ranging from 000 to 6 (e.g., 000 to 0, 000 to 00, 00 to 0, 0 to 3, 0 to 2, 0 to 1, 1 to 3, 1 to 2, 2 to 3, 3 to 6, 3 to 5, 3 to 4, 4 to 6, 4 to 5, 5 to 6, or any range bounded by two values with the range of 000 to 6), e.g., as measured according to ASTM D937-07 when using an unworked sample or ASTM D217-02 when using an unworked sample. In some embodiments, a probiotic composition has a NLGI consistency grade of 000. In some embodiments, a probiotic composition has a NLGI consistency grade of 00. In some embodiments, a probiotic composition has a NLGI consistency grade of 0. In other embodiments, a probiotic composition has a NLGI consistency grade of 1. In other embodiments, a probiotic composition has a NLGI consistency grade of 2. In other embodiments, a probiotic composition has a NLGI consistency grade of 3. In other embodiments, a probiotic composition has a NLGI consistency grade of 4. In other embodiments, a probiotic composition has a NLGI consistency grade of 5. In other embodiments, a probiotic composition has a NLGI consistency grade of 6. Compositions of the disclosure are generally softer at higher temperatures and more firm at lower temperatures. Compositions of different consistency can be prepared, for example, by varying the amount of one or more thickeners (e.g., one or more thickeners described in Section 5.2.2.2) and/or by varying the type and/or amount of a bulk component (e.g., one or more bulk components as described in Section 5.2.2.1). For example, the consistency of a probiotic composition can be increased by increasing the amount of thickener and/or substituting a high viscosity oil in place of a low viscosity oil.

Preferably, probiotic compositions of the disclosure have a consistency which is not “runny” over the temperature range of 10° C. to 70° C., 10° C. to 60° C., 10° C. to 50° C., or 10° C. to 40° C. “Runniness” of a probiotic composition at a given temperature can be measured by loading a 300 ml cartridge sized to fit a standard caulking gun and having a ½ inch inner diameter nozzle opening with 300 ml of the probiotic composition and positioning the cartridge vertically with the nozzle pointing down while maintaining the temperature. A composition which is not runny at a given temperature will not drip from the nozzle when the cartridge is positioned vertically for 10 minutes.

In addition to consistency and “runniness,” viscosity of a probiotic composition can also be evaluated. For example, the viscosity of a probiotic composition can be tested using a rotational viscometer, e.g., a RM 100 Plus Viscometer (Lamy Rheology Instruments). In some embodiments, a probiotic composition of the disclosure has a viscosity between 30,000 cP to 780 M cP at 20° C. (e.g., 30,000 to 1 McP, 30,000 to 500,000 cP, 30,000 to 250,000 cP, 50,000 to 500,000 cP, 50,000 to 250,000 cP, 100,000 cP to 500,000 cP, 100,000 to 250,000 cP, or 250,000 to 500,000 cP).

The probiotic compositions of the disclosure can “stick” to the vaginal wall of a non-human animal. “Stickiness” of a probiotic composition can be measured, for example, by a bioadhesion force assay. An exemplary in vitro bioadhesion assay is described in El-Kamel and El-Khatib, 2006, Drug Delivery, 13(2):143-148, incorporated herein by reference. In some embodiments, a probiotic composition of the disclosure has a bioadhesive force ranging from 5,000 to 20,000 dyne/cm² (e.g., 5,000 to 15,000, 5,000 to 10,000, 10,000 to 20,000, or 10,000 to 15,000 dyne/cm²) as measured by the in vitro bioadhesion assay described in El-Kamel and El-Khatib, 2006, Drug Delivery, 13(2):143-148.

The probiotic compositions of the disclosure can in some embodiments have a specific gravity at 23° C. ranging from 1.1 to 1.2, e.g., 1.10, 1.10, 1.11, 1.12, 1.13, 1.14, 1.15, 1.16, 1.17, 1.18, 1.19, or 1.20, or any range bounded by any of the forgoing values. Specific gravity of a probiotic composition can be determined by (1) calibrating a measuring device (e.g., a measuring scoop) by weighing the measuring device when empty and weighing the measuring device when filled with distilled water at 23° C. to determine the weight of the empty measuring device and the weight of the distilled water, (2) filling the calibrated measuring device with the probiotic composition at 23° C., (2) weighing the filled measuring device, (3) subtracting the weight of the empty measuring device from the weight of the filled measuring device to determine the weight of the probiotic gel in the measuring device, and (4) dividing the weight of the probiotic gel by the weight of the distilled water to obtain the specific gravity of the probiotic gel.

Preferably, the probiotic compositions of the disclosure are free of animal proteins (e.g., milk proteins). Probiotic compositions free of animal proteins are preferred from a regulatory standpoint.

### 5.2.1. Probiotic Bacteria

The probiotic compositions of the disclosure can contain one or more strains of bacteria (e.g., one strain), and generally contain two or more (e.g., two strains), three or more (e.g., three strains), or four or more (e.g., four strains) strains of bacteria. Without being bound by theory, it is believed that probiotic compositions having two or more strains of bacteria can be superior to probiotic compositions having a single strain of bacteria because communal relationships formed among strains of probiotic bacteria are believed to be beneficial to the establishment and/or maintenance of a healthy vaginal microbiome.

Probiotic compositions comprising two or more strains of bacteria can contain an equal amount of each strain or can contain different amounts of the different strains (on a CFU basis). For a probiotic composition containing strains of bacteria that grow at significantly different rates, a composition can be made that includes, for example, more of the strain(s) that grow at a slower rate(s) and less of the strain(s) that grow at a faster rate(s).

For, example, for a composition having two strains of bacteria, each strain can account for 10% to 90% of the total amount of bacteria (on a CFU basis) (e.g., one strain can be 10% to 20%, 10% to 30%, 10% to 40%, 20% to 30%, 20% to 40%, 20% to 50%, 30% to 40%, or 40% to 50% of the total, with substantially all of the remainder being the second strain), provided that the amounts of the two strains are selected so that the sum of the amounts of the two strains does not exceed 100%. Preferably, probiotic compositions are free of contaminating bacteria (e.g., airborne bacteria that are unintentionally introduced into a probiotic composition during manufacture or contaminating bacteria from a fermentation used to produce a strain of bacteria for a probiotic composition). For a probiotic composition having two strains of probiotic bacteria and which is free of contaminating bacteria, the sum of the amounts of the two strains will equal 100% of the total. However, if a small amount of contaminating bacteria are present (e.g., less than 0.5%, less than 0.4%, less than 0.3%, less than 0.2%, or less than 0.1% of the total bacteria on a CFU basis), the sum of the amounts of the two strains will be slightly less than 100% of the total amount of bacteria in the composition (e.g., if a probiotic composition has 0.1% of contaminating bacteria and two non-contaminating strains, the sum of the amounts of the two strains will be 99.9% of the total).

As another example, for a composition having three strains of bacteria, each of the three strains can account for 10% to 50% of the total amount of bacteria in the probiotic composition (on a CFU basis) provided that the total amount for the three strains together does not exceed 100% (e.g., each of the strains can account of about ⅓ of the total; one of the strains can account for about 50% of the total and each of the other two strains can account for about 25% of the total; one of the strains can account for about 20% of the total, one of the strains can account for 30% of the total, and one of the strains can account for about 50% of the total). In some embodiments, each of the three strains in present in an amount which is at least 5% of the total amount of bacteria in the composition on a CFU basis (e.g., at least 5%, at least 10%, at least 15%, at least 20%, or at least 25%). For compositions which are free of contaminating bacteria, the sum of the amounts of the three strains will equal 100% of the total bacteria. However, if a small amount of contaminating bacteria are present (e.g., less than 0.5%, less than 0.4%, less than 0.3%, less than 0.2%, or less than 0.1% of the total bacteria on a CFU basis), the sum of the amounts of the three strains will be slightly less than 100% of the total (e.g., if a probiotic composition has 0.1% of contaminating bacteria and three non-contaminating strains, the sum of the amounts of the three strains will be 99.9% of the total).

Generally, the one or more strains of bacteria are native to the vagina of healthy members of the species of animal for which the probiotic composition is intended to be used. For example, one or more strains of bacteria can be isolated from the vaginal tract of healthy dairy cows and cultured to provide an amount of bacteria that can be used to make a probiotic composition of the disclosure. In some embodiments, the bacteria included in a probiotic composition of the disclosure comprise or consist of one or more strains of bacteria that are native to healthy pregnant animals. In other embodiments, the bacteria included in a probiotic composition of the disclosure comprise or consist of one or more strains of bacteria that are native to healthy non-pregnant animals.

The probiotic compositions of the disclosure preferably include one or more strains of bacteria that are not native to the gastrointestinal tract of the species of animal for which the probiotic composition is intended to be used. Thus, in some embodiments, a probiotic composition of the disclosure can include one or more strains of bacteria comprising or consisting of one or more strains of bacteria that are native to the vagina of the species of animal for which the probiotic composition is intended to be used, but not native to the gastrointestinal tract of the animal.

In various embodiments, the strains of bacteria comprise or consist of bacteria having one, two, three, four, five six, seven, or eight of the following characteristics:
a) non-hemolytic, gram-positive, catalase-negative, and capable of growing under anaerobic conditions;
b) capable of growing in a pH range of 3 to 9 (e.g., 4 to 9, 4 to 8, 4 to 7, 4 to 6, 4 to 5, 5 to 9, 5 to 8, 5 to 7, 5 to 6, 6 to 9, 6 to 8, or 7 to 8);
c) capable of fast growth at different temperatures (e.g., at temperatures ranging from 15° C. to 45° C., 15° C. to 40° C., 20° C. to 45° C., or any value within any range, for example 20° C., 25° C., or 39° C.);
d) capable of aggregating, either among members of the same strain (auto-aggregation) or with members of a genetically different strain (co-aggregation);
e) lactic acid producing;
f) hydrogen peroxide producing;
g) ability to adhere to vaginal mucus;
h) produce a bacteriocin that inhibits one or more pathogenic strains of bacteria.

In some embodiments, the one or more strains of bacteria comprise or consist of one or more strains of Lactic acid bacteria (LAB). The bovine vagina is typically neutral to slightly alkaline (e.g., pH of approximately 7 to 8), and LAB can provide an acid vaginal pH through the production of lactic acid (and, for some species, H₂O₂), which can make it difficult for pathogenic bacterial to colonize. LAB that can be included in the probiotic compositions of the disclosure include strains of Abiotrophia, Aerococcus, Bifidobacterium, Carnobacterium, Enterococcus, Lactobacillus, Lactococcus, Leuconostoc, Oenococcus, Pediococcus, Streptococcus, Tetragenococcus, Vagococcus, Weissella, or a combination thereof.

In some embodiments, the one or more strains of bacteria included in a probiotic composition of the disclosure comprise or consist of one or more strains of Abiotrophia.

In some embodiments, the one or more strains of bacteria included in a probiotic composition of the disclosure comprise or consist of one or more strains of Aerococcus.

In some embodiments, the one or more strains of bacteria included in a probiotic composition of the disclosure comprise or consist of one or more strains of Bifidobacterium.

In some embodiments, the one or more strains of bacteria included in a probiotic composition of the disclosure comprise or consist of one or more strains of Carnobacterium.

In some embodiments, the one or more strains of bacteria included in a probiotic composition of the disclosure comprise or consist of one or more strains of Enterococcus.

In some embodiments, the one or more strains of bacteria included in a probiotic composition of the disclosure comprise or consist of one or more strains of Lactobacillus. Exemplary species of Lactobacillus that can be included in a probiotic composition include L. sakei, L. reuteri, L. rhamnosus, L. buchneri, L. mucosae, L. gasseri, L. delbrueckii, or a combination thereof. In some embodiments, a probiotic composition of the disclosure comprises one or more strains of L. sakei, for example L. sakei FUA 3089 (Genbank accession no. GQ222408.1).

In some embodiments, the one or more strains of bacteria included in a probiotic composition of the disclosure comprise or consist of one or more strains of Lactococcus.

In some embodiments, the one or more strains of bacteria included in a probiotic composition of the disclosure comprise or consist of one or more strains of Leuconostoc.

In some embodiments, the one or more strains of bacteria included in a probiotic composition of the disclosure comprise or consist of one or more strains of Oenococcus.

In some embodiments, the one or more strains of bacteria included in a probiotic composition of the disclosure comprise or consist of one or more strains of Pediococcus. In some embodiments, a probiotic composition of the disclosure comprises one or more strains of P. acidilactici, for example P. acidilactici FUA 3138 (Genbank accession no. GQ222409.1) and/or P. acidilactici FUA 3140 (Genbank accession no. GQ222392.1).

In some embodiments, the one or more strains of bacteria included in a probiotic composition of the disclosure comprise or consist of one or more strains of Streptococcus.

In some embodiments, the one or more strains of bacteria included in a probiotic composition of the disclosure comprise or consist of one or more strains of Tetragenococcus.

In some embodiments, the one or more strains of bacteria included in a probiotic composition of the disclosure comprise or consist of one or more strains of Vagococcus.

In some embodiments, the one or more strains of bacteria included in a probiotic composition of the disclosure comprise or consist of one or more strains of Weissella.

In a preferred embodiment, the one or more strains of bacteria comprise or consist of L. sakei FUA 3089, P. acidilactici FUA 3138 and P. acidilactici FUA 3140.

The one or more strains of bacteria are preferably included in the probiotic composition in dried form. Various methods for drying bacteria are known in the art and can be used. See, e.g., Corcoran et al., 2004, Journal of Applied Microbiology, 96:1024-1039; Morgan et al., 2006, Journal of Microbiological Methods 66:183-193; Santivarangkna et al., 2007, Biotechnol. Prog. 23:302-315; Meng et al., 2008, Food Chemistry 106:1406-1416. In some embodiments, the one or more strains of bacteria are spray-dried. In other embodiments, the one or more strains of bacteria are lyophilized. The drying can be performed in the presence of a prebiotic, for example a prebiotic described in Section 5.2.2.3, or in the absence of a prebiotic.

### 5.2.2. Non-Aqueous Base

The probiotic compositions of the disclosure comprise a non-aqueous base. The non-aqueous base can comprise a single component (e.g., a single bulk component identified below) or multiple components (e.g., one or more bulk components together with one or more thickeners). Generally, probiotic compositions of the disclosure comprise a multi-component non-aqueous base. For example, a non-aqueous base can include an oil and a thickener that acts as a gelling agent. Some components that can be included in probiotic compositions of the disclosure can have more than one function, and such components can be included in a probiotic composition for one, more than one, or all of its functions. For example, some components can function as both a thickener and a prebiotic. Identification of a component as a thickener does not preclude it from also being considered a prebiotic and vice versa.

### 5.2.2.1. Bulk Components

Generally, a non-aqueous base of a probiotic composition comprises one or more inert components that together comprise a substantial amount of the probiotic composition by weight (e.g., 50% to 90%, 50% to 80%, 50% to 70%, 50% to 60%, 60% to 90%, 60% to 80%, 60% to 70%, 70% to 90%, 70% to 80%, or 80% to 90% of the probiotic composition by weight). Such substances can be referred to as “bulk components.” Exemplary substances that can be used as bulk components, alone or in combination, are described in Sections 5.2.2.1.1 and 5.2.2.1.2. For avoidance of doubt, it should be understood that the substances described in Sections 5.2.2.1.1 or 5.2.2.1.2 can be included in a probiotic composition in any suitable amount.

### 5.2.2.1.1. Non-Water Soluble Bulk Components

A non-aqueous base can include one or more bulk components that are not water soluble or poorly water soluble, for example one or more oils, one or more waxes, one or more fatty substances such as cocoa butter, or a combination thereof. Such components can be used, for example, to prepare a probiotic composition that is resistant to dissolution by body fluids and/or that dissolves slowly in the presence of body fluids.

A non-aqueous base can include one or more oils. Preferably, the one or more oils are food grade oils. The one or more oils can be natural or synthetic. The one or more oils can be plant-derived or non-plant-derived (e.g., mineral oil). Blends of oils can also be used (e.g., a blend of one or more natural oils and one or more synthetic oils, a blend of one or more plant-derived oils and one or more non-plant-derived oils, a blend of plant-derived oils, etc.).

Plant-derived oils can be from a genetically modified plant (“GMO plants”) or a non-GMO plant. Preferably, one or more plant-derived oils, all of which are from non-GMO plants are used. Plant-derived oils that can be used include soybean oil, borage seed oil, flaxseed oil, evening primrose oil, canola oil, safflower oil, sunflower oil, grapeseed oil, sesame oil, hemp seed oil, pumpkin seed oil, and combinations thereof. In some embodiments, the non-aqueous base comprises soybean oil. In some embodiments, the non-aqueous base comprises borage seed oil. In some embodiments, the non-aqueous base comprises flaxseed oil. In some embodiments, the non-aqueous base comprises evening primrose oil. In some embodiments, the non-aqueous base comprises canola oil. In some embodiments, the non-aqueous base comprises safflower oil. In some embodiments, the non-aqueous base comprises sunflower oil. In some embodiments, the non-aqueous base comprises grapeseed oil. In some embodiments, the non-aqueous base comprises sesame oil. In some embodiments, the non-aqueous base comprises hemp seed oil. In some embodiments, the non-aqueous base comprises pumpkin seed oil.

A non-aqueous base can comprise one or more waxes, for example in combination with one or more oils. Waxes that can be used include beeswax and paraffin wax.

A non-aqueous base can comprise one or more fatty substances, for example, cocoa butter or a cocoa butter substitute. Cocoa butter substitutes include synthetic triglycerides and triglycerides from plant oils (e.g., from palm oil, palm kernel oil, coconut oil, or a combination thereof). Fatty substances can be used, for example, for making a suppository (see, de Villiers, 2009, “Suppository Bases,” Chapter 24 of A Practical Guide to Contemporary Pharmacy Practice, 3^{rd} Edition).

### 5.2.2.1.2. Water Soluble Components

A non-aqueous base can include one or more bulk components that are water soluble, for example glycerinated gelatin and hydrophilic polymers such as polyethylene glycols (PEGs). Such components can be used to prepare a probiotic composition that dissolves in the presence of body fluids. Mixtures of PEGs having different molecular weights can be used. For example, a non-aqueous base can comprise a combination of PEGs, e.g., as described in Table 24.2 of de Villiers, 2009, “Suppository Bases,” Chapter 24 of A Practical Guide to Contemporary Pharmacy Practice, 3^{rd} Edition, the contents of which are incorporated herein by reference. Water soluble bulk components can be used, for example, for preparing a probiotic composition in the form of a suppository that does not melt at body temperature but dissolves in body fluid.

### 5.2.2.2. Thickeners

Non-aqueous bases comprising one or more oils generally include one or more thickeners. Similarly, non-aqueous bases comprising one or more water soluble components described in Section 5.2.2.1.2 can include one or more thickeners. Thickeners can function as gelling agents, forming a gel with the other components of the probiotic composition.

Thickeners that can be used include silicon dioxide, calcium sulfate, sodium sulfate, magnesium sulfate, one or more oligosaccharides, one or more polysaccharides, one or more emulsifiers, silica, one or more bentonite clays, sodium alginate, whey protein, or a combination thereof. Other thickeners that are known in the art can also be used.

In some embodiments, a probiotic composition of the disclosure comprises silicon dioxide. In addition to acting as a thickener, silicon dioxide can also scavenge moisture, thereby performing multiple functions in a probiotic composition.

In some embodiments, a probiotic composition of the disclosure comprises calcium sulfate, sodium sulfate, magnesium sulfate, or a combination thereof.

In some embodiments, a probiotic composition comprises one or more oligosaccharides. Exemplary oligosaccharides include fructooligosaccharides (FOS) (e.g., from wheat, rye, chicory, asparagus, Jerusalem artichokes, soybeans, or another plant).

In some embodiments, a probiotic composition comprises one or more polysaccharides. Exemplary polysaccharides include starches, dextrins, maltodextrins, pullulan and pullulan derivatives, agarose, and combinations thereof. In some embodiments, a probiotic composition comprises pullulan and/or one or more pullulan derivatives. Exemplary pullulan derivatives that can be used include esterified pullulan, etherified pullulan, hydrogenated pullulan, sulfated pullulan, chlorinated pullulan, cholesterol substituted pullulan, and fatty acid substituted pullulan (see, Park and Khan, 2009, Chapter 21 in Handbook of Hydrocolloids, 2^{nd} Edition, pp. 592-614, the contents of which are incorporated herein by reference).

Exemplary starches that can be used include corn starch, potato starch, wheat starch, oat starch, barley starch, rice starch, sorghum starch, a legume starch (e.g., from a pea or a bean), tapioca, and combinations thereof. In a preferred embodiments, a probiotic composition of the disclosure comprises corn starch, optionally in combination with silicon dioxide. Starches suitable for inclusion in a probiotic composition of the disclosure include native starches, modified starches, and combinations thereof. Modified starches are known in the art (see, e.g., Bertolini, ed., 2009, Starches: Characterization, Properties, and Applications, CRC Press, Boca Raton, Fla.) and include chemically treated starches, alkali and/or acid washed starches, enzymatically hydrolyzed starches, bleached starches, esterified starches, cross-linked starches, ionized starches, and oxidized starches.

In some embodiments, a probiotic composition of the disclosure comprises one or more one or more emulsifiers, such as a lecithin (e.g., from egg, soybean, rapeseed, cottonseed, or sunflower). Probiotic compositions comprising lecithin preferably contain plant-derived lecithin such as soybean, rapeseed, cottonseed, or sunflower lecithin.

In some embodiments, a probiotic composition of the disclosure comprises one or more bentonite clays (e.g., sodium bentonite, calcium bentonite, potassium bentonite, or a combination thereof).

In some embodiments, a probiotic composition of the disclosure comprises sodium alginate.

In some embodiments, a probiotic composition of the disclosure comprises a whey protein (e.g., a whey protein isolate, a whey protein concentrate, a whey protein hydrolysate, or a combination thereof).

### 5.2.2.3. Prebiotics

Probiotic compositions of the disclosure can optionally further include one or more prebiotics. Inclusion of a prebiotic can help to reduce the amount of time after in vivo application of a probiotic composition to an animal before the bacteria in the probiotic composition “wake up” and begin metabolism, growth, reproduction, and colonization the vaginal tract. A short lag time is desirable so that the bacteria in the probiotic composition have the opportunity to colonize the vaginal tract of the animal before the components of the probiotic composition are expelled from the animal due to contractions, urination, etc.

Exemplary prebiotics include monosaccharides, disaccharides, oligosaccharides (e.g., as described in Section 5.2.2.2), polysaccharides (e.g., as described in Section 5.2.2.2), fermentation products, and combinations thereof. In some embodiments, one or more prebiotics are selected that also function as thickeners.

In some embodiments, a probiotic composition of the disclosure comprises one or more monosaccharides. Exemplary monosaccharides that can be used include dextrose, fructose, and galactose. In one embodiment, the probiotic composition comprises dextrose. The one or more monosaccharides can be in anhydrous or hydrated form. Preferably, the one or more monosaccharides are in anhydrous form.

In some embodiments, a probiotic composition of the disclosure comprises one or more disaccharides. Exemplary disaccharides that can be used include sucrose, lactose, maltose, and trehalose. In one embodiment, the probiotic composition comprises sucrose (e.g., as powdered sugar). In another embodiment, the probiotic composition comprises trehalose.

In some embodiments, a probiotic composition of the disclosure comprises one or more oligosaccharides, e.g., an FOS, as described in Section 5.2.2.2.

In some embodiments, a probiotic composition of the disclosure comprises one or more fermentation products. Fermentation products can include unutilized nutrients from a fermentation broth (e.g., a MRS fermentation broth) such as amino acids, peptides, carbohydrates, and vitamins, dead cell debris, and products produced by the microorganisms during fermentation, such as bacteriocins.

The one or more fermentation products are preferably spray-dried fermentation products, however other dried fermentation products (e.g., produced by lyophilization, oven drying, fluid bed drying, or other drying processes) can also be used. The one or more fermentation products can include one or more fermentation products made from a fermentation that produced a strain of bacteria that is included in the probiotic composition. For example, a probiotic composition comprising Lactobacillus sakei and Pediococcus acidilactici can in some embodiments include a L. sakei fermentation product and/or a P. acidilactici fermentation product. Thus, a fermentation product that might otherwise be discarded can be included in a probiotic composition as a prebiotic, thereby reducing waste and/or the cost to produce the probiotic composition. Additionally, and without being bound by theory, it is believed that fermentation products made from a fermentation that produced a strain of bacteria included in the probiotic composition may contain compounds such as bacteriocins that inhibit pathogenic microorganisms. For example, P. acidilactici FUA3138 and FUA3140 produce the bacteriocin pediocin AcH/PA-1. Wang et al., 2013, BMC Microbiology 13:19.

### 5.2.2.4. Additional Additives

Probiotic compositions of the disclosure can optionally further include one or more additives, for example one or more biologically active ingredients. Such additives can include vitamins, minerals, antioxidants, and carotenoids. Carotenoids have immunostimulatory effects on mucosal membranes, and can be included in reduced and/or oxidized form. An exemplary carotenoid that can be included in a probiotic composition is beta-carotene.

### 5.2.3. Exemplary Probiotic Compositions

An exemplary probiotic composition of the disclosure comprises the following components:
a) one or more strains of bacteria (e.g., L. sakei FUA 3089, P. acidilactici FUA 3138 and P. acidilactici FUA 3140);
b) dextrose (e.g., anhydrous dextrose);
c) sucrose (e.g., powdered sugar);
d) corn starch;
e) fructooligosaccharide;
f) silicon dioxide;
g) soybean oil (e.g., non-GMO soybean oil); and
h) one or more fermentation products (e.g., a Lactobacillus sakei fermentation product and a Pediococcus acidilactici fermentation product.

In a further embodiment, the probiotic composition described in the foregoing paragraph comprises:
a) the one or more strains of bacteria;
b) 2% to 6% dextrose, by weight of the composition;
c) 3% to 9% sucrose, by weight of the composition;
d) 2% to 5% corn starch, by weight of the composition;
e) 4% to 12% fructooligosaccharide, by weight of the composition;
f) 10% to 20% silicon dioxide, by weight of the composition;
g) 50% to 70% soybean oil, by weight of the composition; and
h) a Lactobacillus sakei fermentation product and a Pediococcus acidilactici fermentation product, which together are 0.5% to 3% of the composition by weight,
   provided that the amounts of components (a)-(h) are selected so that the sum of the weights of the components does not exceed 100%.

In a further embodiment, the probiotic composition described in the foregoing paragraph comprises:
a) the one or more strains of bacteria;
b) about 4% dextrose, by weight of the composition;
c) about 6% sucrose, by weight of the composition;
d) about 3.5% corn starch, by weight of the composition;
e) about 8% fructooligosaccharide, by weight of the composition;
f) about 14% silicon dioxide, by weight of the composition;
g) about 63% soybean oil, by weight of the composition; and
h) a Lactobacillus sakei fermentation product and a Pediococcus acidilactici fermentation product, which together are about 1% of the composition by weight.

Another exemplary probiotic composition of the disclosure comprises the components described in the foregoing paragraph, each component present in an amount which is ±20% from the amounts described in the foregoing paragraph, provided that the total amounts of all components sum to no more than 100% of the weight of the composition (e.g., the amount of dextrose can range from about 3.2% to about 4.8%). In another exemplary probiotic composition of the disclosure comprises the components described in the foregoing paragraph, each component present in an amount which is ±15% from the amounts described in the foregoing paragraph, provided that the total amounts of all components sum to no more than 100% of the weight of the composition. In another exemplary probiotic composition of the disclosure comprises the components described in the foregoing paragraph, each component present in an amount which is ±10% from the amounts described in the foregoing paragraph, provided that the total amounts of all components sum to no more than 100% of the weight of the composition. In another exemplary probiotic composition of the disclosure comprises the components described in the foregoing paragraph, each component present in an amount which is ±5% from the amounts described in the foregoing paragraph, provided that the total amounts of all components sum to no more than 100% of the weight of the composition.

The exemplary compositions described in this Section and comprising L. sakei FUA 3089, P. acidilactici FUA 3138 and P. acidilactici FUA 3140 are suitable for administration to cows (e.g., dairy cows). L. sakei FUA 3089, P. acidilactici FUA 3138 and P. acidilactici FUA 3140 can be substituted with strains of bacteria native to the vaginal tracts of other animals, e.g., horses, to make compositions suitable for administration to such animals.

The exemplary compositions described in this Section can also be modified to omit and/or substitute components (e.g., to substitute oils such as those described in Section 5.2.2.1 and/or thickeners such as those described in 5.2.2.2). The skilled person can make such omissions and/or substitutions, and adjust the amounts of the remaining and/or substituted components accordingly so as to provide alternative compositions of similar consistency. For example, an exemplary probiotic composition having the following components omits dextrose and a fermentation product:
a) one or more strains of bacteria;
b) about 6% sucrose, by weight of the composition;
c) about 5% corn starch, by weight of the composition;
d) about 8% fructooligosaccharide, by weight of the composition;
e) about 15% silicon dioxide, by weight of the composition; and
f) about 66% soybean oil, by weight of the composition.

Another exemplary probiotic composition has the following components:
a) one or more strains of bacteria;
b) one or more fermentation products (e.g., less than 1% by weight of the composition);
c) about 6% sucrose (e.g., powdered sugar), by weight of the composition;
d) about 4% corn starch, by weight of the composition;
e) about 8% fructooligosaccharide, by weight of the composition;
f) about 15% silicon dioxide, by weight of the composition; and
g) about 66% soybean oil, by weight of the composition.

Additional exemplary compositions are described in the Examples.

### 5.3. Probiotic Products, Kits, and Systems

### 5.3.1. Probiotic Products

In one aspect, the disclosure provides probiotic products comprising a probiotic composition of the disclosure packaged within a container. The container can be a single use or multi-use container. For example, the container can be a single use syringe having an amount of probiotic composition suitable for a single administration (e.g., 5 ml to 50 ml, 5 ml to 30 ml, 5 ml to 20 ml, 5 ml to 10 ml, 10 ml to 50 ml, 10 ml to 30 ml, 10 ml to 20 ml, 20 ml to 50 ml, 20 ml to 50 ml or 30 ml to 50 ml). Alternatively, the container can be a multi-use container having an amount of probiotic composition suitable for multiple administrations. Multi-use containers can be advantageous in commercial farming, where it may be desirable to administer the probiotic composition to multiple animals at a time.

Multi-use containers for packaging gels are known in the art and include cartridges suitable for use with an applicator gun (e.g., a caulking gun or bovine dosing gun such as Nordson P/N 7660620), and metered syringes (e.g., Dial-a-Dose® syringes, Nordson). Different sized cartridges and applicator guns are available commercially (e.g., 300 ml and 850 ml cartridges and correspondingly sized applicator guns) and are suitable for use with the probiotic compositions of the disclosure. Such cartridges typically have a nozzle, which can have a seal that needs to be removed prior to the first use. For example, the seal can comprise foil or a closed end portion of the nozzle that can be pierced or cut off prior to use. Cartridges can further have a removable cap for closing the cartridge between uses.

Off the shelf cartridges and as well as custom sized cartridges can be used. Cartridges can be filled with varying amounts of a probiotic composition, for example an amount in the range of 10 ml to 1000 ml (e.g., 10 ml 100 ml, 100 ml to 200 ml, 200 ml to 1000 ml, 200 to 500 ml, 200 ml to 400 ml, 400 ml to 600 ml, 500 ml to 800 ml, or 500 ml to 1000 ml). A cartridge can be partially filled with the probiotic composition (e.g., a cartridge having a 850 ml capacity can be filled with 800 ml of the probiotic composition) or fully filled with the probiotic composition. In some embodiments, probiotic product of the disclosure comprises a cartridge (e.g., a 10.5 ounce cartridge or 300 ml cartridge) filled with approximately 300 ml of a probiotic composition.

Metered syringes are commercially available in various sizes (e.g., 30 ml, 60 ml, 80 ml, and 100 ml) and are typically filled with a smaller volume compared to a cartridge, for example 50 ml to 100 ml (e.g., approximately 60 ml or approximately 80 ml). A syringe can be partially filled with the probiotic composition or fully filled with a probiotic composition.

In another aspect, the disclosure provides probiotic products comprising a probiotic composition of the disclosure packaged within a capsule. Various types of capsules are known in the art and can be used. For example, the capsule can be a gelatin-based capsule (e.g., as described in WO/1984/004675) or a pullulan-based capsule (e.g., as described in WO/2005/105051). In some embodiments, a capsule suitable for administration to cows, horses, pigs, and other livestock contains 5 to 20 ml of a probiotic composition (e.g., 5 ml to 15 ml, 10 ml to 20 ml, 5 ml to 10 ml, 10 ml to 15 ml, or 15 ml to 20 ml).

In another aspect, the disclosure provides probiotic products comprising a probiotic composition of the disclosure in the form of a suppository. A suppository suitable for administration to cows, horses, pigs, and other livestock can contain, for example, 5 to 20 ml of a probiotic composition (e.g., 5 ml to 15 ml, 10 ml to 20 ml, 5 ml to 10 ml, 10 ml to 15 ml, or 15 ml to 20 ml). Suppositories can be in any suitable shape, for example bullet or torpedo shaped, round oval shaped, elongated oval shaped, tampon shaped, teardrop shaped, cone shaped, or any other suppository shape known in the art.

Capsules and probiotic compositions that are in the form of suppositories can be packaged within a container or package, for example, a bottle, tub, box, blister pack, or wrapper.

### 5.3.2. Probiotic Kits

In one aspect, the disclosure provides probiotic kits comprising (i) a probiotic product of the disclosure, where the probiotic product comprises a probiotic composition packaged within a container, and (ii) an applicator tube having a proximal end dimensioned for attachment to an end of the container. For example, the applicator tube can comprise a tube dimensioned for attachment to a nozzle of a cartridge. The applicator tube is useful for administering the probiotic composition into the vaginal tract of an animal, and the length of the tube can be appropriately selected based on the anatomy of the species of animal. For example, an applicator tube for administering a probiotic product to a large animal such as a cow will generally be longer than an applicator tube for administering a probiotic product to a smaller animal such as a sheep or goat. For a cow, the total length of an applicator tube can be, for example, 6 to 15 inches long (e.g., 6 to 12 inches, 6 to 9 inches, 9 to 18 inches, 9 to 15 inches, 9 to 12 inches, 12 to 18 inches, or 12 to 15 inches).

While applicator tubes are generally capable of being attached and removed from the container of a probiotic product as described herein, applicator tubes which are fixed to the container are also envisioned (e.g., a syringe or cartridge can comprise an integrated applicator tube, for example having the length and inner diameter of an applicator tube as described in this Section). Applicator tubes that are capable of being attached and removed from the container have the advantage of reusability. For example, such an applicator tube can be used to administer a probiotic composition from a first cartridge and then reused to administer a probiotic composition from a second cartridge. A removable applicator tube can further comprise an integrated clip for securing the applicator tube to a container. The clip can comprise, for example, a hose clamp (e.g., similar to a Herbie Clip®) or strap (e.g., similar to a cable tie) which is fixed to the applicator tube. When closed, the clip can provide a clamping force around the circumference of the applicator tube, helping to secure the applicator tube to the cartridge.

The inner diameter of an applicator tube can be constant throughout. Alternatively, an applicator tube can have a larger inner diameter at the proximal end (i.e., the end that attaches to the container) and a smaller inner diameter at the distal end (i.e., the end which is intended to be inserted into the vaginal tract of the animal during administration). The inner diameter can decrease uniformly from the proximal end to the distal end or, alternatively, the inner diameter can decrease non-uniformly from the proximal end to the distal end. For example, the diameter can decrease from the proximal end to the distal end by one or more step downs (e.g., one step down, two step downs, or three step downs).

The outer dimensions of the applicator tube can be constant throughout, or alternatively, the outer dimensions can be larger at the proximal end and smaller at the distal end. The outer dimensions can decrease at a uniform rate or at a non-uniform rate (e.g., when the applicator tube has one or more step downs).

In some embodiments, the inner diameter of the proximal end of the applicator tube is ¼ to ¾ inches, for example ½ inches. The inner diameter of the distal end can in some embodiments be smaller, for example, ⅛ inches to ½ inches (e.g., ¼ inches to ⅜ inches). In some embodiments, the inner diameter of the distal end of the applicator tube is ¼ inches or ⅜ inches.

The distal end of the applicator tube is preferably smooth so that the applicator tube does not injure the animal when used. For example, applicator tubes comprising an acrylic or polycarbonate rod at the distal end can be burnished to smooth the end of the rod.

The applicator tube can be constructed of a single material or, alternatively, different sections of the applicator tube can be constructed of different materials. For example, the proximal end of the applicator tube can comprise a flexible material (e.g., vinyl tubing), while the distal end can comprise a material which is less flexible than the material used to make the proximal end. In some embodiments, the material used to make the distal end of the applicator tube is a rigid material (e.g., acrylic rod, polycarbonate rod or other polymeric material). The lengths of the applicator tube made of flexible and/or rigid material can vary (e.g., they can be selected based upon the species of animal for which the applicator tube is intended to be used). For example, a length of flexible material of 3 to 5 inches, e.g., 3 inches, 4 inches, or 5 inches, and a length of rigid material of 5 to 13 inches, e.g., 7 inches to 11 inches, 8 to 10 inches, 5 inches, 6 inches, 7 inches, 8 inches, 9 inches, 10 inches, 11 inches, 12 inches, or 13 inches can be used to make an applicator tube useful for administering a probiotic composition to a cow. Shorter lengths of either or both materials can be used to make applicator tubes for smaller animals.

A kit can comprise one applicator tube per probiotic product (e.g., a single applicator tube and a single cartridge) or can comprise a different ratio of applicator tubes to probiotic products (e.g., less than one applicator tube per cartridge). For example, a kit can comprise two applicator tubes and six cartridges.

In another aspect, the disclosure provides a kit comprising a capsule or suppository of the disclosure and an applicator for the capsule or suppository. Such applicators are known in the art (see, e.g., U.S. Pat. No. 4,990,136).

### 5.3.2.1. Exemplary Applicator Tubes

Exemplary applicator tubes suitable for administering a probiotic composition to a dairy cow are described in this section. The applicator tubes can optionally be scaled up or down for use with smaller or larger animals (e.g., in length only, or in length and another dimension such as inner diameter).

In a first exemplary applicator tube, the applicator tube comprises a proximal end made from ¾ inch outer diameter (OD)×½ inch inner diameter (ID) tubing (e.g., vinyl tubing) and ½ inch OD×⅜ inch ID rod (e.g., acrylic or polycarbonate) at the distal end. The rod can be positioned and fixed (e.g., by glue) in an end of the tubing, thus providing a step down from a ½ inch ID to ⅜ inch ID. The tubing can be 3 to 5 inches long, for example 3 inches. The rod can be 5 to 13 inches long, for example 9 inches.

In a second exemplary applicator tube, the applicator tube comprises a proximal end made from ¾ inch outer diameter (OD)×½ inch inner diameter (ID) tubing (e.g., vinyl tubing), ½ inch OD×⅜ inch ID tubing (e.g., vinyl tubing) between the proximal and distal ends, and a ⅜ inch OD×¼ inch ID rod (e.g., acrylic or polycarbonate) at the distal end. The proximal end tubing can be 3 to 5 inches long, for example 3 inches. The tubing between the proximal end and distal end can be 1 to 4 inches long, for example 1.5 inches. The rod can be 5 to 13 inches long, for example 9 inches. One end of the ½ inch OD×⅜ inch ID tubing can be positioned in and fixed in one end of the ¾ inch OD×½ inch ID tubing, and the rod can be positioned and fixed in the other end of the ½ inch OD×⅜ inch ID tubing, thereby providing two step downs (from ½ to ⅜ inch and from ⅜ inch to ¼ inch).

In a third exemplary applicator tube, the applicator tube comprises a proximal end made from ¾ inch outer diameter (OD)×½ inch inner diameter (ID) tubing (e.g., vinyl tubing), a ½ inch OD×⅜ inch ID rod (e.g., acrylic or polycarbonate), and a ⅜ inch OD×¼ inch ID rod (e.g., acrylic or polycarbonate) at the distal end. One end of the ½ inch OD×⅜ inch ID rod can be positioned in and fixed in one end of the ¾ inch OD×½ inch OD tubing, and the ⅜ inch OD×¼ inch ID rod can be positioned and fixed in the other end of the ½ inch OD×⅜ inch ID rod, thereby providing two step downs (from ½ to ⅜ inch and from ⅜ inch to ¼ inch). The final step down can function as a “choke,” increasing the velocity at which the probiotic composition exits the applicator tube. The proximal end tubing can be 3 to 5 inches long, for example 3 inches. The rod between the proximal end and distal end can be 5 to 13 inches long, for example 9 inches. The rod at the distal end can be ½ inch to 9 inches long, for example 1.5 inches.

### 5.3.2.2. Additional Kit Components

The probiotic kits of the disclosure can optionally further comprise an applicator gun sized for the container of the kit. Suitable applicator guns are known in the art and include bovine dosing guns (e.g., Nordson P/N 7660695) and caulking guns. The applicator gun preferably dispenses a fixed volume with each trigger pull (e.g., 5 ml to 50 ml, 5 ml to 40 ml, 5 ml to 40 ml, 5 ml to 30 ml, 5 ml to 20 ml, 5 ml to 10 ml, 10 ml to 50 ml, 10 ml to 40 ml, 10 ml to 30 ml, 10 ml to 20 ml, 20 ml to 50 ml, 20 ml to 40 ml, 20 ml to 30 ml, 30 ml to 50 ml, 30 ml to 40 ml, or 40 ml to 50 ml), thereby allowing the user to easily dispense the desired volume of probiotic composition. In some embodiments, the applicator gun dispenses 5 ml with each trigger pull. In some embodiments, the applicator gun dispenses 10 ml with each trigger pull.

Kits of the disclosure can optionally further comprise one or more clips for securing a removable applicator tube to a cartridge. Exemplary clips include hose clamps, e.g., a metal or plastic hose clamp such as a Herbie Clip® plastic hose clamp and cable ties.

Kits of the disclosure can optionally further comprise wipes for cleaning an applicator tube between uses (e.g., between administrations to separate animals) or disposable sleeves that can be placed over the applicator tube and discarded after use. Cleaning wipes are known in the art and can include one or more sanitizers, for example one or more of alcohol (e.g., ethanol and/or isopropanol), sodium hypochlorite (bleach), and didecyl dimethyl ammonium chloride.

### 5.3.3. Systems

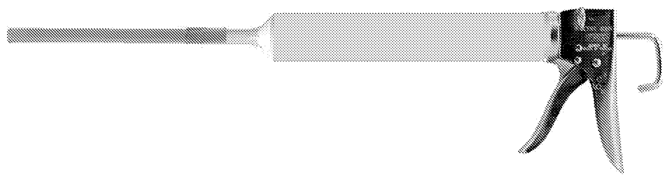

The disclosure further comprises a system comprising a probiotic kit of the disclosure configured for use. For example, a system can comprise a probiotic product with an applicator tube attached to the container of the probiotic product (e.g., an applicator tube attached to a metered syringe or an applicator tube attached to a cartridge). An exemplary system comprising an applicator tube attached to a cartridge is shown in FIG. 1. Systems of the disclosure can further comprise a probiotic product positioned in an applicator gun. Thus, in some embodiments, a system of the disclosure comprises an applicator gun loaded with a cartridge having an attached applicator tube, and having an amount of the probiotic composition in the cartridge. In some embodiments, the system comprises a 300 ml cartridge. Systems of the disclosure can be assembled from kits of the disclosure at any time prior to use, and in some embodiments are assembled immediately before use.

### 5.4. Uses

The disclosure provides methods of using the probiotic compositions, probiotic products, kits, and systems of the disclosure for introducing one or more strains of bacteria to the vagina of a non-human animal. Introducing the one or more strains of bacteria to the vagina can have a prophylactic and/or a therapeutic effect. For example, the therapeutic effect can comprise treating a uterine and/or urogenital infection caused by one or more of Escherichia coli, Truperella pyogenes, Fusobacterium necrophorum, and Bacteroides melaminogenicus.

The non-human animal can be, for example, a domesticated animal. The domesticated animal can be a ruminant such as a cow, sheep, or goat, or a non-ruminant such as a horse or pig. In some embodiments, the non-human animal is a cow (e.g., a dairy cow). Cows can be of the species Bos taurus or Bos indicus. Exemplary breeds of Bos taurus that can be treated according to the methods of the disclosure include Holstein, Brown Swiss, Guernsey, Ayrshire, Jersey, Red and White, or Milking Shorthorn breeds, and mixed breeds of any of the foregoing. Exemplary Bos indicus breeds include Sahiwal and Gir breeds. In other embodiments, the animal is a pig. In yet other embodiments, the animal is a horse.

In the methods described herein, the probiotic composition (when not packaged in a capsule or in the form of a suppository) is preferably administered via an applicator tube (e.g., as described in Section 5.3.2). The applicator tube (preferably lubricated prior to use) can be inserted in the vagina to an appropriate depth for the species of animal. For example, for administration of a probiotic composition to a cow, an applicator tube can be inserted 3 to 12 inches into the vagina (e.g., 3 to 9 inches, 3 to 6 inches, 6 to 12 inches, 6 to 9 inches, or 9 to 12 inches). Capsules and suppositories can be likewise be inserted in the vagina to an appropriate depth for the species of animal, e.g., manually or with an applicator.

In one aspect, the disclosure provides methods for introducing one or more strains of bacteria to the vagina of a non-human animal comprising administering an amount of a probiotic composition of the disclosure to the vagina of the animal. The probiotic composition can be administered, for example, to an animal before, during, or following a period of stress (e.g., pregnancy, labor, injury, or infection) or before, during, or following a course of antimicrobial therapy (e.g., treatment with an antibacterial antibiotic or an antifungal medication). For example, a probiotic composition of the disclosure can be administered to an animal before, during, or following treatment with an oral antibiotic (sulfamethazine, oxytetracycline, or sulfadimethoxine) or antibiotic administered by injection (e.g., ceftiofur, penicillin, ampicillin, oxytetracycline, erythromycin, tylosin, sulfadimethoxine, amoxicillin, tilmicosin, florfenicol, sulfamethazine, or enrofloxacin). The probiotic composition can be administered, for example, after the last treatment with an antibiotic (e.g., within about 1 month of the last treatment, about 4 weeks after the last treatment, about 3 weeks after the last treatment, about 2 weeks after the last treatment, or about 1 week after the last treatment), or even during treatment with an antibiotic. The bacteria in the probiotic composition can help reestablish or supplement the normal bacterial microflora of the reproductive tract that can be reduced during a period of stress or antimicrobial therapy. When normal reproductive tract microflora is present the probiotic composition can help to support the immune system and health.

In one aspect, the disclosure provides methods for treating a uterine infection (e.g., metritis, endometritis, or pyometra) in a non-human animal comprising administering a therapeutically effect amount of a probiotic composition of the disclosure to the vagina of the animal.

In another aspect, the disclosure provides methods for lowering the risk of contracting a uterine infection (e.g., metritis, endometritis, or pyometra) in a non-human animal comprising administering a therapeutically effect amount of a probiotic composition of the disclosure to the vagina of the animal.

In another aspect, the disclosure provides methods for treating a urogenital infection (e.g., a urinary tract infection or vaginitis) in a non-human animal comprising administering a therapeutically effect amount of a probiotic composition of the disclosure to the vagina of the animal.

In another aspect, the disclosure provides methods for lowering the risk of contracting a urogenital infection (e.g., a urinary tract infection or vaginitis) in non-human animal comprising administering a therapeutically effect amount of a probiotic composition of the disclosure to the vagina of the animal.

In another aspect, the disclosure provides methods for promoting the establishment or maintenance of a healthy vaginal microbiome in a non-human animal comprising administering a therapeutically effect amount of a probiotic composition of the disclosure to the vagina of the animal.

In another aspect, the disclosure provides methods of increasing the amount of colostrum and/or increasing the immunoglobulin content of colostrum produced by a non-human animal (e.g., a dairy cow), comprising administering an amount of a probiotic composition of the disclosure to the vagina of a pregnant animal prior to labor (e.g., one or more time prior to labor according to a pre-partum administration regimen described herein). Without being bound by theory, it is believed that administration of the probiotic composition to a pregnant animal prior to labor can increase the amount of colostrum and increase the immunoglobulin content by improving the immune health of the animal.

The methods of the disclosure can comprise administering the probiotic composition one time or more than one time. For example, a probiotic composition can be administered at least once (e.g., once), at least twice (e.g., twice), at least three times (e.g., three times) or more than three times (e.g., four times, five times, or six times). A single administration can comprise administering of an amount of the probiotic composition as a single dose (e.g., by a single trigger pull of an applicator gun), or as multiple doses (e.g., by two or more trigger pulls of an applicator gun). Administrations can be separated by a period of time ranging from about 1 day to about 1 month (e.g., 1 day, about 2 days, about 3 days, about 4 days, about 5 days, about 6 days, about 1 week, about 2 weeks, about 3 weeks, about 4 week, or about 5 weeks). In some embodiments, multiple administrations are separated from each other by about 1 week (e.g., 5 days to 9 days, 6 days to 8 days, or 7 days).

The amount of bacteria administered in each administration can range, for example, from 10⁹ to 10¹³ CFU (e.g., 10⁹ to 10¹², 10⁹ to 10¹¹, 10⁹ to 10¹⁰, 10¹⁰ to 10¹³, 10¹⁰ to 10¹², 10¹⁰ to 10¹¹, 10¹¹ to 10¹³, 10¹¹ to 10¹², 10¹² to 10¹³, 1 billion to 8 billion, 1 billion to 6 billion, 1 billion to 4 billion, 1 billion to 2 billion, 3 billion to 10 billion, 3 billion to 8 billion, 3 billion to 6 billion, 3 billion to 4 billion, 5 billion to 10 billion, 5 billion to 8 billion, 5 billion to 6 billion, 7 billion to 10 billion, 7 billion to 8 billion or 8 billion to 10 billion). In some embodiments, each administration contains 4 billion to 5 billion CFU (e.g., about 4.5 billion CFU). The concentration of bacteria in a probiotic composition can be selected so that a desired volume of probiotic composition can be administered at each administration while administering a desired amount of bacteria. For example, in some embodiments 10 ml (a suitable administration volume for cows, horses, pigs, and other livestock) of a probiotic composition containing at least 0.45 billion CFU per 1 ml is used so that at least 4.5 billion CFUs of bacteria are administered per administration.

In some embodiments, the animal can be an animal that is pregnant when the probiotic composition is administered for the first time. In other embodiments, the animal has given birth less than one month (e.g., less than 4 weeks, less than 3 weeks, less than 2 weeks, or less than 1 week) before administration of the probiotic composition for the first time. Administration of the probiotic composition before and/or after labor can be used to accelerate involution, accelerate resumption of ovarian cyclicity, and/or reduce the number of days open (the number of days from calving to conception) in the animal following labor. Administration of the probiotic composition prior to labor can also reduce the incidence of retained placenta. Administration of the probiotic composition before and/or after calving can also promote an increase in milk production in dairy cows following calving.

Administration regimens for pregnant animals can comprise one or more pre-partum administrations (e.g., one, two, three, four or more than four pre-partum administrations). In some embodiments, an administration regimen for a pregnant animal comprises one pre-partum administration, for example about 4-6 weeks prior to the expected calving date (e.g., about 4 weeks, about 5 weeks, or about 6 weeks), about 2-4 weeks prior to the expected calving date, or about 1-2 weeks prior to the expected calving date (e.g., about 1 week or about 2 weeks). In other embodiments, an administration regimen for a pregnant animal comprises two pre-partum administrations. The first pre-partum administration can be, for example, about 4 to 6 weeks prior to the expected date of labor (e.g., about 4 weeks, about 5 weeks, or about 6 weeks), or about 2 to 4 weeks prior to the expected date of labor (e.g., about 2 weeks, about 3 weeks, or about 4 weeks). The second pre-partum administration can be, for example, about 2 to 4 weeks prior to the expected date of labor (e.g., about 2 weeks, about 3 weeks, or about 4 weeks) or about 1 to 2 weeks prior to the expected date of labor (e.g., about 1 week or about 2 weeks). In an embodiment, the first pre-partum administration is about 4 to 6 weeks prior to the expected date of labor (e.g., about 4 weeks, about 5 weeks, or about 6 weeks) and the second pre-partum administration is about 1 to 2 weeks prior to the expected date of labor (e.g., about 1 week or about 2 weeks).

Administration regimens for pregnant animals can further comprise one or more post-partum administrations (e.g., one, two, three, four or more than four post-partum administrations). Similarly, administration regimens for animals that have recently given birth and which are administered the probiotic composition for the first time following labor can comprise one or more post-partum administrations (e.g., one, two, three, four or more than four post-partum administrations).

In some embodiments, the methods comprise one post-partum administration. In some embodiments, the methods comprise two post-partum administrations. In some embodiments, the methods comprise three post-partum administrations. In some embodiments, the methods comprise four post-partum administrations. The first post-partum administration can be, for example, within about 1 week of labor (e.g., on the day of labor, the day after labor, 2 days after labor, 3 days after labor, 4 days after labor, 5 days after labor, 6 days after labor, 7 days after labor, 1 to 3 days after labor, 2 to 4 days after labor, 3 to 5 days after labor, 4 to 6 days after labor, 5 to 7 days after labor, 5 to 9 days after labor, or 6 to 8 days after labor. Subsequent post-partum administrations can be separated from the previous administration, for example, by about 1 day, about 2 days, about 3 days, about 4 days, about 5 days, about 6 days, about 7 days, or by about 1 week (e.g., 5 days to 9 days, 6 days to 8 days, or 7 days). In some embodiments, a first post-partum administration is within about 1 week of labor (e.g., on the day of labor, the day after labor, 2 days after labor, 3 days after labor, 4 days after labor, 5 days after labor, 6 days after labor, 7 days after labor, 1 to 3 days after labor, 2 to 4 days after labor, 3 to 5 days after labor, 4 to 6 days after labor, 5 to 7 days after labor, 5 to 9 days after labor, 6 to 8 days after labor, or 7 days after labor), and a second post-partum administration is about 2 weeks post-partum (e.g., 12 to 16 days after labor, 13 to 15 days after labor, or 14 days after labor).

### 6. EXAMPLES

### 6.1. Example 1: Safety Study

A safety study is conducted in open and pregnant (>7 months) Holstein dairy cows to assess the safety of a probiotic composition of the disclosure.

### 6.1.1. Materials and Methods

The probiotic composition used in this study contains the probiotic bacteria L. sakei FUA 3089, P. acidilactici FUA 3138, and P. acidilactici FUA 3140. A 10 ml dose of the composition contains at least 4.5 billion total CFU of the bacteria. The components of the probiotic composition are shown in Table 1.

TABLE 1

| Component | Amount (wt %) |
|---|---|
| Powdered Sugar | 6.30 |
| Corn Starch | 4.73 |
| Fructooligosaccharide (FOS) | 8.1 |
| Silicon dioxide | 14.95 |
| Soy Oil | 65.766 |
| Pediococcus acidilactici 3138 | 0.033 |
| Pediococcus acidilactici 3140 | 0.030 |
| Lactobacillus sakei | 0.091 |

The probiotic composition is administered to four open and four pregnant cows according to the study protocol shown in Table 2. Each dose is 10 ml.

TABLE 2

| | | Number of doses | | | | |
|---|---|---|---|---|---|---|
| | Study | Study | Study | Study | Study | Total |
| Animal | Day 0 | Day 1 | Day 7 | Day 14 | Day 21 | Doses |
| Open Cow 1 | Observe | 1X | 1X | 1X | Observe | 3 |
| Open Cow 2 | Observe | 2X | 2X | 2X | Observe | 6 |
| Open Cow 3 | Observe | 3X | 3X | 3X | Observe | 9 |
| Open Cow 4 | Observe | 2X | 5X | 2X | Observe | 9 |
| Pregnant Cow 1 | Observe | 1X | 1X | 1X | Observe | 3 |
| Pregnant Cow 2 | Observe | 2X | 2X | 2X | Observe | 6 |
| Pregnant Cow 3 | Observe | 3X | 3X | 3X | Observe | 9 |
| Pregnant Cow 4 | Observe | 2X | 5X | 2X | Observe | 9 |

Cows are observed twice daily for straining, pain, or other discomfort.

A vaginoscopy is performed on SD 0, SD 3, SD 7, SD 9, SD 12 SD 14, SD 16, SD 18, and SD 21. The vaginal mucus is scored according to the 4-point scoring system of Williams et al., 2005, Theriogenology. 63(1):102-17 (0=clear mucus; 1=mucus containing flecks of pus; 2=discharge containing less than 50% pus; 3=discharge containing more than 50% pus).

Appetite and milk production are monitored daily. Weight is monitored weekly.

### 6.1.2. Results

The probiotic composition is found to be well tolerated and safe.

### 6.2. Example 2: Microbial Re-Faunation Kinetics Study

A study is conducted in open and pregnant (>7 months) Holstein dairy cows to assess vaginal microbial re-faunation kinetics following administration of a probiotic composition of the disclosure.

### 6.2.1. Materials and Methods

The probiotic composition used in this study has the same composition as the probiotic composition described in Example 1. The probiotic composition (10 ml) is administered on Day 1 of the study to four open and two pregnant cows. Samples of vaginal mucus 16s ribosome rRNA profiling and culturing are obtained five times throughout the study as shown in the Table 3.

TABLE 3

| | Study | Study | Study | Study | Study | Study |
|---|---|---|---|---|---|---|
| Animal | Day 0 | Day 1 | Day 2 | Day 7 | Day 14 | Day 21 |
| Open Cow 1 | Sample | 1X | Sample | Sample | Sample | Sample |
| Open Cow 2 | Sample | 1X | Sample | Sample | Sample | Sample |
| Open First Calf | Sample | 1X | Sample | Sample | Sample | Sample |
| Heifer 1 | | | | | | |
| Open First Calf | Sample | 1X | Sample | Sample | Sample | Sample |
| Heifer 2 | | | | | | |
| Dry Pregnant | Sample | 1X | Sample | Sample | Sample | Sample |
| Cow 1 | | | | | | |
| Dry Pregnant | Sample | 1X | Sample | Sample | Sample | Sample |
| Cow 2 | | | | | | |

### 6.2.2. Results

Administration of the probiotic composition is found to promote establishment and/or maintenance of a healthy vaginal microbiome.

### 6.3. Example 3: Multi-Herd Study

A study is conducted pregnant Holstein dairy cows to assess the prophylactic and/or therapeutic effect of administration of a probiotic composition of the disclosure.

### 6.3.1. Materials and Methods

The probiotic composition used in this study has the same composition as the probiotic composition described in Example 1. The probiotic composition (10 ml) is administered to pregnant cows about 4 to 6 weeks prior to the expected calving date and about 2 weeks prior to the expected calving date. Following calving, the probiotic composition (10 ml) is administered about 2 weeks and about 4 weeks after calving.

### 6.3.2. Results

Administration of the probiotic composition is found to reduce the incidence of uterine infections (e.g., metritis), urogenital infections, accelerate uterine involution, accelerate resumption of ovarian cyclicity, and reduce the number of days open.

### 6.4. Example 4: Tolerance Study

A study was conducted to evaluate the local and overall tolerance to a probiotic composition containing a live consortia of three natural GRAS listed lactic acid bacteria in a ready-to-use multi-dose formulation.

### 6.4.1. Materials and Methods

### 6.4.1.1. Formulation

The probiotic compositions used in this Example were ready-to-use cGMP 21 CFR Part 11 compliant ISO 9001 manufactured formulations, provided in 300 cc HDPE cartridges. Each 10 mL dose contained 5 billion CFU's of total lactic acid producing microorganisms (LAB) (Lactobacillus sakei, FUA 3089, Pediococcus acidilactici FUA 3140, Pediococcus acidilactici FUA3138) in non-aqueous base of soy oil, corn starch, silicone dioxide, fructooligosaccaharide (FOS), and powdered sugar or a combination of powdered sugar and dextrose. The components of the probiotic composition without dextrose are shown in Table 2. The formulations were designed to promote post administration tolerance, retention, colonization and LAB viability.

TABLE 2

| Component | Amount (wt %) |
|---|---|
| Powdered Sugar | 6.30 |
| Corn Starch | 4.73 |
| Fructooligosaccharide (FOS) | 8.1 |
| Silicon dioxide | 14.95 |
| Soy Oil | 65.766 |
| Pediococcus acidilactici 3138 | .033 |
| Pediococcus acidilactici 3140 | .030 |
| Lactobacillus sakei | .091 |

### 6.4.1.2. Study Design

Six pregnant cows averaging 227 days pregnant (50% Lactation 3, 50% Lactation 1) and six lactating cows averaging 26 Days in Milk (50% Lactation 2 and 50% Lactation 3) were randomly assigned to one of four groups to receive the probiotic composition on Study Day (SD) 1, 7 and 14 at the following dose volumes:
Group 1: 10 mL administered on SD 1, 7 and 14 (three dry cows; three lactating cows);
Group 2: 20 mL administered on SD 1, 7 and 14 (one dry cow; one lactating cow);
Group 3: 30 mL administered on SD 1, 7 and 14 (one dry cow; one lactating cow);
Group 4: 20 mL administered on SD 1, 50 mL on SD 7, and 20 mL on SD 14 (one dry cow; one lactating cow).

The administration schedule was the same for both lactating and dry cows.

### 6.4.1.3. Administration

The probiotic composition was applied intravaginally using a system comprising the cartridge containing the probiotic composition, an applicator gun, and an 11 inch long, ½ inch OD by ⅜ inch ID clear acrylic tube that attached to the nose of the cartridge with a 3 inch long, ½ inch ID by ¾ inch OD clear PCV hose. Prior to each administration, the tube was sanitized with a sanitizing wipe (Clorox Corp pre-moistened 1.4% Hydrogen Peroxide Wipes size 4.3×8.4 inches). Approximately 9 inches of the applicator tube was inserted into the vagina and each pull of the dose trigger provided 10 mL of the probiotic composition.

### 6.4.1.4. Animal Evaluation

The 21-day study evaluated the animals' responses to administration of the probiotic composition and included local tolerance post administration, evaluating irritation/discomfort and/or straining. Daily evaluations noted any external vaginal discharge including external evidence of the gel formulation back leakage. Vaginoscopy evaluations (VE) were conducted on SD 0, 3, 7, 9, 12, 14, 16, 18 and 21, and included monitoring for any adverse events. In addition, daily attitude, appetite, and rumen fill scores were obtained. Physical Exams, body weight and rectal temperature on measured on SD 0 and SD 21. Milk production in the lactating cows was measure from PM milking on SD 0, 3, 7, 9, 12, 15, 17, 19 and 21.

The test article administrator evaluated the degree of restraint needed to apply the probiotic composition using a Restrain Score scale of 0-3 with 0 being use of a feed bunk head locks only and no other restraint. A score of 1 was defined for when the addition of moderate tail restraint was needed. A score of 2 indicated the need for significant tail restraint and a score of 3 indicated that additional restraint was required. The three administration periods (SD 1, 7, 14) for the 12 cows provided a total of 36 encounters.

The immediate cow reaction (“Cow Reaction Score”) was scored by the test article administrator as 0 for none, 1 for some uneasiness and 2 for mild straining, 3 for significant straining and 4 if greater discomfort was noted.

The administrator also rated ease of administration with a Score of 0 for easy and 1 if a problem was noted.

Prior to first administration of the probiotic composition and for 21 consecutive days the cows were evaluated for Attitude (0 for Normal—bright alert responsive, 1 for Mild Depression 2 for Moderate to Marked Depression).

Appetite was scored via watching feeding for at least 2 minutes, and then separately evaluating Rumen Fill Score on a 1-5 Scale (Zaaijer and Noordhuizen, 2003). Briefly, this evaluates the fill level and shape of the para lumbar fossa. A score of 0 indicates that the fossa cavitates significantly, a score of 1 indicates the fossa cavitates less than a hands width, a score of 3 indicates that the fossa appears relatively even to the transverse processes, a score of 4 indicates that the fossa bulges out and a score of 5 indicates that the fossa bulges and the last rib is not visible.

Straining was evaluated as 0 for None and 1 for any sign of discomfort.

Adverse events were also noted and if present documented.

In addition, external observations for vaginal discharge and gel were made daily. Discharge was scored 0 for none or clear, 1 if mucus was noted with flecks of pus, 2 if discharge was <50% pus and 4 if greater. If blood was observed, it was noted in comments.

Vaginoscopy evaluations (VE) on SD 0, 3, 7, 9, 12, 14, 16, 18 and 21 used a clear plastic 1.5-inch×18-inch tube (Jorgensen Labs). The Investigator Scored 0 if no observations of if clear mucus was observed, 1 if mucus was present with flecks of pus and 2 if the discharge <50% pus and 3 if discharge >50% pus. Comments were noted if blood was present or to note other observations. Gel presence was scored 0 if none was observed, 1 if slight amounts observed, 2 of moderate amounts and 3 if significant amounts were present.

Physical exams inclusive of an overall Clinical Assessment, Appearance, Behavior (to rule out aggressive cows), assessments of Respiratory, Sensory Organs, Urine/Feces, Feet/Legs, Circulatory System, Digestive System, Urogenital System Integument, Musculoskeletal and Nervous system were conducted on SD 0 and 21. Rectal temperature and body weight were also collected and recorded.

### 6.4.2. Results

Restraint scores were 0 for 34/36 encounters and 1 for two encounters. In summary, normal feed bunk head gates provided sufficient restraint 94% of the time.

Gel retention post dosing was scored as complete for 100% of the administrations in both the dry and lactating cattle.

All 36 encounters for cow reaction scored as a 0.

All 36 encounters were scored 0 for ease of administration.

Attitude score was 0 for all subjects on all study days except on SD 2 for a single lactating cow in the group that was administered the 10 mL on SD 1, 7 and 14. She was noted as Straining on Day 2 and had both low Rumen Fill (Score 1) and a purulent vaginal discharge (Score 3). No action was taken, and this resolved in 24 hours and was not observed again.

Appetite was evaluated as 0 (observed at feed bunk) for all days and subjects except for one day in the lactating group when cows could not be observed, as bunks were clean, thus Rumen Fill scores were used. For the dry cows, Rumen Fill scores were 3 or higher except for one cow on SD2. For lactating cows there were 9 observations of Rumen Fill of 2 and 2 observations of Rumen Fill of 1. For the six lactating cows observed daily for 21 day, there was a total of 126 observations with 115 or 91% of the Rumen Fill scores being three or above.

One episode of straining was noted on SD 2 for one lactating cow which had a discharge score of 3 on vaginoscopy. Otherwise no straining was observed on any day in either the Lactating or Dry Groups for any of the administered volumes.

No Adverse Events were noted by the Investigator.

External vaginal discharge was 0 and no gel was observed in the dry cows for all study days and dose levels. For the lactating cows that were administered 10 mL on SD 1, 7 and 14, 2 of the three subjects had a 0 Vaginal Discharge score for all study days. The one subject noted above had a score of 3 observed on 2 separate occasions and a score of 1 on two separate occasions. No external vaginal discharge of gel was noted in the lactating groups that were administered the higher dose levels. One subject (cow ID 8187) had a discharge score of 3 observed on 3 separate occasions, a score of 2 on one observation and a score of 1 on two separate occasions. She remained healthy, milking in the 100 pounds of milk per day range. She was the smallest lactating cow assigned and lost about 4 kg of BW consistent with her production class. A vaginal muco-purulent discharge in an apparently healthy cow is consistent with endometritis

For dry cows, at all dose levels VE Scores were 0 at each evaluation and no gel was observed. For the lactating cows administered 30 mL on SD 1, 7 and 14 and 20 mL on SD 1, 50 mL on SD7 and 20 mL SD 14 VE Scores were 0 and no gel was observed (0). For the lactating cow administered the 20 mL on SD 1, 7 and 14, VE Scores of 1 were noted on VE Days 7, 9, 12 and 21. For the lactating cows administered the 10 mL dose on SD 1, 7 and 14, one subject had VE Scores of 0 and no gel observed. One subject administered the 10 mL dose on SD 1, 7 and 14 had a VE Score of 1 prior to administration on SD1 (0). A VE Scores of 2 on SD 14 and the 1 SD 16, 18 and 21 were noted. The third lactating cow in this group cow noted with the higher external vaginal discharge scores (10 mL on SD 1, 7 and 14) VE Scores on SD 1 prior to administration was 1, and 3 on SD's 12, 14, 16 18 and 21. Gel was observed by VE in only one cow three days after administration. Scope and external discharge scores were not associated.

On SD 0, the dry cow body weights ranged from 484 to 874 kg BW, average of 735.2 kg and on SD 21 body weights ranged from 486.5 kg to 854 kg and averaged of 726.2 kg. The lactating cows' body weight on SD 0 ranged from 534 to 722 kg and averaged 660.2 kg. On SD 21 the lactating cows' body weight ranged from 529.5 kg to 751.5 kg and averaged 652.4 kg.

Milk production for the lactating cows by test article administration is reported Table 3. PM milk production was measured only on 6 days post administration: SD 4, 9, 12, 15, 19 and 21 by the staff of DairyExperts using Waikato MKV Milk Meters (Waikato Milking Systems, Hamilton, New Zealand).

TABLE 3

| | Lactation | SD 0 | SD 4 | SD 9 | SD 12 | SD 15 | SD 19 | SD 21 |
|---|---|---|---|---|---|---|---|---|
| Group | | PM Milk Yield (Lbs) | | | | | | |
| Group I | 3 | 48 | 52 | 44 | 36 | 46 | 49 | 43 |
| (cow ID | | | | | | | | |
| 5769) | | | | | | | | |
| Group I | 3 | 46 | 44 | 43 | 46 | 42 | 46 | 44 |
| (cow ID | | | | | | | | |
| 2415) | | | | | | | | |
| Group I | 2 | 42 | 47 | 58 | 48 | 45 | 50 | 55 |
| (cow ID | | | | | | | | |
| 8187) | | | | | | | | |
| Group 2 | 2 | 27 | 40 | 50 | 40 | 47 | 60 | 45 |
| (cow ID | | | | | | | | |
| 58) | | | | | | | | |
| Group 3 | 2 | 47 | 48 | 48 | 43 | 49 | 44 | 45 |
| (cow ID | | | | | | | | |
| 8119) | | | | | | | | |
| Group 4 | 2 | 49 | 48 | 47 | 64 | 38 | 47 | 43 |
| (cow ID | | | | | | | | |
| 19148) | | | | | | | | |

Physical exams, rectal temperature, and body weight were all were assessed as normal.

### 6.4.3. Discussion

Restraint, and Administration—The only restraint needed were locking feed head locks, with no additional restraint required except on two occasions were some tail restraint was used for one cow. The administrator reported the application was easy with no problems noted with insertion and gel delivery. The intravaginal route was well tolerated and was associated with no adverse effects.

Initial Tolerance Post Administration—There were no immediate cow reactions (uneasiness/discomfort or straining). The immediate evaluation noted that the gel was retained in all subjects. All administrations were reported to have gel retention. No animal reactions were reported in both dry and lactating cows that received the 1× dose as well as for the higher volume doses up to 5×. The intravaginal route and probiotic composition were well tolerated and was associated with no adverse effects.

General Tolerance-Attitude, Appetite, PE, BW Milk Production—In both dry and lactating cows that received the 1× dose as well as for the higher volume doses up to 5×, changes in overall physical exams comparing SD 0 to SD 21 were unremarkable. There was no change in body weight or rectal temperature. For the lactating cattle, milk production showed no changes or trends.

During the period when the probiotic composition was administered there were no observed changes in attitude or trends associated with decreased appetites either evaluated by observation of feed consumption or the evaluation of Rumen Fill. On occasion, there was a day or two when one cow in the lactating group was observed with a Rumen Fill Score of 2 or 1 with no trends or intervention required. This type of observation is common in cows early in lactation. The intravaginal route and test article were well tolerated and was associated with no adverse effects.

External Vaginal Discharge and Vaginoscopy—Dry Cows—Post administration of the test article to dry cows at all dose levels on SD 1, 7 and 14 cows, vaginal discharge was monitored daily for up to 21 days. In these pregnant non-cycling cows who at the start of the study averaged 227 days in calf were 248 days in calf at completion (gestation is 278-287 days). All cows were still in calf at the end of the study and the probiotic composition even at higher dose volumes had no untoward effects in this class of cattle. VE on SD 0, 3, 7, 9, 12, 14, 16, 18 and 21 also showed no discharge or irritation associated with the test article. Neither the VE nor external observations observed any gel. VE permits great surveillance of the mucosal surfaces of the vagina, the cervix can be visualized and discharge that can pool on the floor of the vaginal vault, that may not be seen externally be evaluated. The probiotic composition was well tolerated in non-lactating late gestation cows.

External Vaginal Discharge and Vaginoscopy—Lactating Cows—Post administration of the probiotic composition to open lactating cows on SD 1, 7 and 14, cows vaginal discharge was monitored daily for up to 21 days. These cows were recently fresh (average of 26 DIM at start and 47 DIM at end of the experiment) cycling cows. No external vaginal discharge or gel was noted in the lactating groups that were administered the higher dose level except for one observation of mucus with flecks of pus (Score of 1) in cow 58 on one day. This observation is common as cows begin to cycle. Of the three cows administered 10 mL on SD 1, 7 and 14, two of the three subjects had a 0 Score for external vaginal discharge on all study days. One subject (cow ID 8187) had a score of 3 observed on 3 separate occasions, a score of 2 on one observation and a score of 1 on two separate occasions. She remained healthy, milking in the 100 pounds of milk per day range. She was the smallest lactating cow assigned and lost about 4 kg of BW consistent with her production class. A vaginal muco-purulent discharge in an apparently healthy cow is consistent with endometritis. Cow ID 8187 VE Score prior to test article administration as 1. This cow 8187 showed depression and poor appetite on SD2 but returned to normal on SD3 all consistent with clinical endometritis. Her external vaginal discharge score was typically 0, until SD 21 when discharge was noted (Score 3). This is common when cows cycle and uterine tone returns, and the discharge becomes viable. Having a VE Score of 1 prior to treatment and remaining clinically health and in high production supports the tolerance of the probiotic composition. The intravaginal route and test article were well tolerated and was associated with no adverse effects.

Adverse Events—There were no adverse events noted.

Overall—The administration process required minimal restraint and was considered easy and acceptable with no exceptions. There were no issues with the route of administration of the equipment used. Immediate tolerance by the target animals, dry and lactating dairy cattle, was excellent with no straining or discomfort and the gel was retained by all subjects. Daily monitoring for attitude appetite, rumen fill and external vaginal discharge and well as VE on SD 0, 3, 7, 9, 12, 14, 16, 18 and 21 established supportive tissue tolerance. VE permitted the more compressive evaluation of the vaginal mucosa, cervix and exudate if pooled in the vaginal vault. Body weight and milk production was maintained throughout the study. Physical exams comparing SD 21 to the pre-Study SD 0 supported the broader tolerance of the test article over a range of dose volumes and administration frequencies. There were no adverse events, no late term abortions or other effects on reproduction.

In conclusion, from a perspective of the apparatus and route of administration (intravaginal), the probiotic composition was found to be safe to use and well tolerated by administrator and the animal. Administration was easy, the gel was retained, with minimum restraint. From a perspective of local tolerance at the site of administration (intravaginally), the probiotic composition was well tolerated by the target animals. No straining or cow discomfort was noted. There were no signs of irritation-based on external evaluations or vaginoscopy. From a perspective of overall tolerance to the whole animal, the probiotic composition was again well tolerated as assessed by attitude, appetite, rumen fill, body weight, milk production and pre and last day of treatment physical exams and body temperature plus the absence of adverse reactions.

The tolerance of the probiotic composition, application apparatus, sanitary procedures and route (intervaginal) in this tolerance study at the intended dose volumes, and at higher dose volumes supports the tolerance and safety of the probiotic composition.

### 6.1. Example 5: Single Herd Study

Over the course of about six months, 43 pregnant cows housed on a commercial dairy farm were included in a study of the probiotic compositions of Example 4. Of the 43, 25 cows were not given probiotic compositions and 18 cows were given the probiotic compositions.

The probiotic compositions were administered to dairy cows before calving. After calving, the colostrum quality was measured using a Brix refractometer. Brix refractometers are typically used to measure the amount of sugar in a solution (e.g., in the winemaking industry), but Brix values can also be used to quantify IgG in colostrum. A Brix value of 22% corresponds to 50 mg/mL. Colostrum with a Brix value above this cutoff point can be considered high quality colostrum.

Of the 25 cows that were untreated, 6 cows had colostrum Brix values of 24 or higher with the average being 24.8 Brix. The average for the group of 25 cows was 20.7 Brix.

Of the 18 cows that were treated with the probiotic compositions, 10 cows had colostrum with Brix values of 24 or higher, with the average for the 10 cows being 29 Brix. One cow achieved 32.5 Brix, the highest ever obtained for this farm. The average Brix value for the group of 18 treated cows was 25.2 Brix.

After the dairy farm started administering the probiotic compositions to its cows, a reduction in the incidence of retained placentas and metritis in the herd was also observed. Cows treated with the probiotic compositions, and their calves, outperformed cows not treated with the probiotic compositions, and their calves, on a number of measures, including colostrum quality, calving ease, calf robustness, post-calving recovery, and date to first insemination after calving.

Without being bound by theory, it is believed that the observed results are due to the probiotic compositions' ability to reestablish and/or maintain a healthy reproductive microbiome.

### 6.2. Example 6: Conditions of Use Study

### 6.2.1. Overview

This Conditions of Use Study (COU) is performed to evaluate the impact of a probiotic composition of the disclosure on reproductive health, overall cattle health and performance during the peri-partum, post-partum and time from calving to re confirmation of pregnancy in both first calf heifers and adult lactating cattle across locations (farms) in the US and Canada.

The objectives of this study include the evaluation of:
a) Probiotic composition administration dynamics and host animal tolerance in both dry/springing heifers and lactating cattle (cows and first calf heifers).
b) Probiotic composition vs control impact on calving parameters: including calving ease, if assisted, twins, calf M/F, calf weight, fetal membrane retention, and colostrum quality/standard gravity (S.G.).
c) Probiotic composition vs control impact on vaginal discharge score (0-3 pictorial scale) and rectal temperature on the first 2 weeks (0-14) post-partum and related progression/absence of uterine infections/retained placenta and metritis.
d) Probiotic composition vs control impact on the use of antibiotics, infusions and hormones post-partum.
e) Probiotic composition vs control impact on post-partum disorders including incidence of clinical hypocalcemia (milk fever), displaced abomasum, clinical ketosis, clinical mastitis, pneumonia and dystocia. Cows that have dystocia induced trauma resulting in cervical or vaginal tears are excluded.
f) Probiotic composition vs control subjects milk production through 120-150 days in milk (DIM).
g) Probiotic composition vs control key reproduction measures including: days to first insemination, % heats observed, days observed in heat, insemination rate (IR), conception rate (CR), first service, second service, third service, fourth service plus, pregnancy rate (PR), projected days open, % cows left herd, % cows that left herd for reproduction.

Herds enrolled have at least 80 heifers and or cows calving per month. Control animals are not administered any probiotic composition but receive standard conditions of no intervention. There is one control animal per probiotic treated heifer and adult cow enrolled.

### 6.2.2. Materials and Methods

### 6.2.2.1. Test Article

The probiotic composition of this Example is a ready to use composition with not less than 6.9×10⁸ CFU/gram of total lactic acid producing microorganisms (Lactobacillus sakei, FUA 3089, Pediococcus acidilactici FUA 3140, Pediococcus acidilactici FUA 3138) with excipients. The probiotic composition is supplied in a 300 cc HDPE long nose cartridge (Genesis Plastics). The composition of the formulation is shown in Table 4.

TABLE 4

| Component | Amount (wt %) |
|---|---|
| Powdered Sugar | 6.25 |
| Corn Starch | 3.603 |
| Fructooligosaccharide (FOS) | 8.30 |
| Silicon dioxide | 15.32 |
| Soy Oil | 66.55 |
| Maltodextrin/fermentation | 0.339 |
| supernatant | |
| Pediococcus acidilactici 3138 | 0.119 |
| Pediococcus acidilactici 3140 | 0.12 |
| Lactobacillus sakei 3089 | 0.230 |

Amounts total slightly more than 100% due to variances
in measurement/manufacturing

### 6.2.2.2. Dose Justification

The dose of 10 mL of probiotic composition per administration is the recommended dose level to administer to cattle −14 and −7 days before calving and again +7 and +14 post calving.

Tolerance studies (see Example 4) in dry and lactating cattle showed that the probiotic composition of the tolerance studies was well tolerated with no vaginal irritation, straining or decrease in milk production, no weight loss or other adverse effects.

In this COU study, the probiotic composition is administered to ‘weekly’ cohorts of eligible cows approximately 14 days prior to calving (cattle average gestation is 283 days), which is at an estimated fetal gestation range of 260-269 days. A second administration is made to this cohort 7 days later, and then 7- and 14-days post-partum for a total of four administrations. Cows are grouped and the probiotic composition is administer once per week. Since calving date is not a point date, some cows may only receive one dose of the probiotic composition prepartum and if calving date is delayed a cow or heifer may receive more than two doses of the probiotic composition during the dry period. The schedule assures exposure of the probiotic composition prior to calving given the variability in dates.

### 6.2.2.3. Storage

The test article is stored at a controlled room temperature, 20°-25° C. (68°-77° F.). Based on results of stability studies, it is expected that the active ingredient in the formulation remains stable for the duration of the study in the indicated storage environment. Once a cartridge of the probiotic composition is used on an administration day, it is not be used again in a subsequent week. Each week a fresh cartridge and application tube is used.

### 6.2.2.4. Administration Equipment

Along with the probiotic composition, sufficient number(s) of administration guns are supplied. These are Newborn 407A guns with a lock bolt installed to standardize the dose to 10 mL (2 clicks). The 300-cc cartridge is a bayonet twist mechanism for attachment.

In addition, sufficient 9-11-inch burnished end acrylic administration tubes are provided. These are ½ inch Outer Diameter (OD) and ⅜ inch Internal Diameter (ID) and are attached to a flexible connector to attach to the cartridge. The flexible connector is a 3-inch clear PCV vinyl hose ½ inch ID by ⅜ inch OD. One end attaches to the nose of the cartridge and the other to the administration tube. The connector is secured using a Herbie Clip® at the nose and an EZ Clip™ on the administration end.

### 6.2.2.5. Sanitation Equipment

Clorox Healthcare or similar brand Hydrogen Peroxide Disinfecting Wipes (30824) 6.75×5.75 inch are provided. Alternatively, Clorox Healthcare Bleach Germicidal Wipes (CL030577) or similar, 6×5 inch are provided. Disposable Nitrile gloves are also provided.

### 6.2.2.6. Animals

The animals used in the study are as described in Table 5.

TABLE 5

| | |
|---|---|
| Species/Breed | Bovine. Pregnant adult dairy cattle and pregnant springing heifers at calf |
| | gestational age 260-269 days (14-20 days before expected calving date). |
| | Cattle are intended for dairy production. Breed characteristics are recorded, |
| | if known. No oral or parenteral prophylactic or performance antibiotics |
| | except monensin (feed) from 30 days pre partum to 21 days post-partum |
| | except as noted. Animals requiring treatment can be treated. |
| Justification | The species used in this study is a target animal species for this test article. |
| Source | Local sources; The producers' complete address with zip code, is |
| | documented in the study records. Data is included in the final report for the |
| | study. |
| Gender | Females |
| Age at Initial | Springing Heifers at least 20 months of age |
| Dose | Adult Cattle <8 years of age |
| Administration | |
| Weight of Test | At least 1000 pounds if Holstein type and 600 pounds if Jersey |
| Animals at | |
| Initial Dose | |
| Administration | |
| Unique Animal | Unique animal identification number in use at the farm and approved by the |
| Identification | sponsor |
| Number of | Herds have at least 80 cows and/or heifers planned to calve each month for |
| Animals | at least 4 months. Herds have up to 1500 cows (probiotic composition and |
| Required | controls) with 200 enrollees minimum. |

### 6.2.2.7. Animal Health

### 6.2.2.7.1. Preventative Medicine/Concurrent Medications

Treatments prior to starting treatment with the probiotic composition (at dry off etc.) or after probiotic composition administration begins (e.g., vaccinations, anthelmintic) are documented in the Cow Med Records. Records are part of the Dairy Comp 305 or DART or other Farm Record system, or a separate paper record. Enrolled cows administered the probiotic composition or control receive all treatments as other herd mates, except for no oral or parenteral prophylactic or performance antibiotics except monensin (feed) from 30 days pre partum to 21 days post-partum except as noted. On days 0-14 post-partum treatment for metritis requires the metritis treatment definition of Rectal Temperature >103.1° F. (39.5° C.) and a vaginal discharge score of >2.

### 6.2.2.7.2. Humane Care of Animals

Any study animal that becomes moribund or terminally ill during the course of the study is culled and or humanely terminated at the discretion of the Owner/Herdsman or local veterinarian. Euthanasia, if required, is achieved according with AVMA Guidelines for the Euthanasia of Animals: 2013 Edition.

Terminated animals or animals found dead are necropsied, if necessary, in an attempt to determine the cause of death. If needed, the decision as to whether or not tissues are collected for histopathology and/or samples for microbiology is made by the Study Director in consultation with the farm's Veterinarian.

### 6.2.2.8. Inclusion/Exclusion Criteria

### 6.2.2.8.1. Inclusion Criteria

Animals included meet criteria in Table 5. Pregnant Heifers and Pregnant Adult cows are enrolled approximately 20 days prior to their expected calving date. Gestation length is assumed to be 283 days. Cows/heifers are enrolled in weekly cohorts beginning approximately 260 to 269 days pregnant, which is about 14-20 days prior to expected calving date. Holstein type heifers are at least 20 months of age and >1000 pounds.

Subjects are only included if determined to be healthy based on a ‘general’ physical examination, and the calf being of the correct gestation age. Enrollees have four working teats, no evidence of mastitis, are of sufficient body weight (BW) as judged by body condition score (BCS >2.0 out of 5) to have a high likelihood of remaining in the herd for a complete lactation.

The following are recorded on the cow/heifer's electronic or paper record if available: unique farm ID, projected calving date (last breeding date), reasonable likelihood of being pregnant and with of late gestation (ballottement of flank) parity, age, previous calving date (cows), previous DIM/305 production (cows), BCS at enrollment, lameness (yes/no), assurance of four working quarters, general health (e.g., treatments or vaccines), no oral or parenteral antibiotics except dry treatment last 30 days except oral monensin.

### 6.2.2.8.2. Exclusion Criteria

Animals that are determined to be physically unsuitable for use in the study are excluded (e.g., injured or clinically ill). Animals with equivocal health results, concurrent disease, fractious nature are not included in the study. The reason(s) for exclusion are documented in the study records.

### 6.2.2.8.3. Post-Inclusion Removal Criteria

Any animal that receives the probiotic composition or control is considered on Study once enrolled no animal is replaced if culled, removed or dies. Animals removed during the study due to adverse health conditions are documented.

Abnormal findings are recorded by the observer and an Adverse Event Record completed by the Study Director or designee.

### 6.2.2.8.4. Cohort Grouping

For efficient herd management cows are grouped into Cohorts. A cohort is a group of subjects who share a defining characteristic, in this case their projected calving date.

Each week dry cows and springing heifers projected to calve in 14-20 days are screened to assure they are study eligible, and if eligible they receive the probiotic composition or control (no probiotic composition) on the set predetermined study day, one set day for all in the cohort per week. For example, this can be a Thursday. This initial cohort has their second treatment 7 days later. Since calving date has a degree of randomness, post-partum cows that calve receive the probiotic composition or control starting at the next the probiotic composition administration day if they are at least 5 days fresh. This is then repeated at the probiotic composition administration day a week later. Subjects that calve later may overlap to a third pre-partum probiotic composition administration. Post-partum, all subjects have at least 2 probiotic composition administrations.

### 6.2.2.9. Animal Housing and Environment

The animals are owned and housed on commercial farms. No separate facilities are needed. Study cows, both those receiving probiotic composition and control cattle, may be housed with other cattle. A description of the dry cow, up close, fresh pen and early production group housing is provided including type of structures, flooring, ventilation, stalls etc. The facilities have locking type head gates or similar for the administration of the probiotic composition to dry and lactating cows and for the assessment of fresh cows for 0-21 days to obtain rectal temperature and discharge scores.

Daily minimum and maximum outdoor temperature and relative humidity are recorded when possible. The light cycle is the natural (outdoor) light cycle for time of year the study is conducted unless otherwise noted.

### 6.2.2.10. Animal Husbandry

### 6.2.2.10.1. Acclimation

Springing heifers and cows eligible for the study are placed in the dry cow lot or up-close groups the day of initial probiotic composition administration or control treatment, but have been part of the herd for at least 30 days.

### 6.2.2.10.2. Feed and Water

Animals are fed standard diets for dry and fresh cows and heifers at each farm.

The animals are allowed ad libitum access to fresh potable water from automatic watering devices. Water consumption is not measured nor recorded.

Feed consumption is not measured and like water consumption is observed as part of the daily observations, and abnormalities are recorded (Daily Observations).

There are no known contaminants in the diet that would interfere with this study, therefore no specified contaminants have been identified nor acceptance levels set. There are no known contaminants in the water believed to be present at levels that may interfere with the study.

### 6.2.2.11. Study Design

### 6.2.2.11.1. Experimental Design

This is a clinical study and utilizes a randomized design with data from first calf heifers and cows analyzed separately and combined if appropriate. At each study site, the experimental design is a split plot with parity class (heifers or cows) as the whole plot factor and treatment as the split plot factor.

The whole plot design is a completely randomized design with a one-way treatment structure. The subplot experimental design is a generalized randomized block design with one-way treatment structure with blocking based on predicted calving date. Animal is the experimental unit for treatment with animals at each study site enrolled based on predicted calving dates.

Data from the multiple sites is combined and treatment by location interactions is determined.

The clinical phase is conducted at 2-4 commercial farms. There are no bioanalytical assessments.

### 6.2.2.11.2. Sample Size

For acute metritis, observed within 14 days post-partum using vaginal discharge and rectal temperature as detailed in the Excenel FOI (NADA140-890), a sample size of about 100 treated and 100 control cows yields the power to detect a difference at 0.05.

For reproduction indices based on the data from Ferguson and Skidmore, 2013, J. Dairy Sci., 96:1269-1289, reproduction end points such as conception rate or days open, given an alpha of 0.5 and a non-inferiority margin (delta of 15%) with 80% power using a one-sided T-test in a two group study, about 200 cows per treatment group are required. With attrition from cows not remaining to 120-150 DIM adding 20% yields, 250 cows per group are needed with no more than 40% of data from one site given a minimum of 4 sites (locations).

### 6.2.2.11.3. Withdrawal Groups

No milk discard or meat withdrawal is needed. Manufacture of the ready-to-use probiotic composition is cGMP 21 CFR Part 11 compliant ISO 9001. It does not require FDA CVM approval or INAD authorization nor authorization by USDA APHIS CVB.

### 6.2.2.11.4. Blocking Factors

Each weekly cohort has separate blocks of springing heifers and cows.

### 6.2.2.11.5. Masking

At each study location the personnel administering the probiotic composition or managing control is different personnel than those evaluating discharge and rectal temperature. All individuals making health, vaginoscopy, and reproduction assessments are blinded to treatment.

### 6.2.2.11.6. Randomization Procedures

### 6.2.2.11.6.1. Allocation of Animals to Treatment Groups

Starting at least 20 days before the initiation of the study, defined as the first day subjects are administered the probiotic composition or control, the herdsman or study coordinator provides a list of all eligible pregnant and springing heifers due to calve that meet the eligibility (inclusion-exclusion) requirements and have calves that are at gestational age between 260 and 269 days. This is provided via access to DC305, DART Records or a list/spreadsheet providing ID and parity. This list is generated weekly or projected over many weeks until enrolment is complete.

### 6.2.2.11.6.2. Randomization

Randomization is performed using the KUTOOLS add-on random number generator function in Excel®. An Excel® spreadsheet is prepared using the following procedure:
1) By location, a separate list of study-eligible animal ID numbers for heifers and cows is entered into Column 1 ranking those closest to gestation day 269 in decreasing order to those further out.
2) The treatment groups (probiotic composition or control) are assigned to each pair closest in in ranked gestational age for both heifers and cows using the random number generator. NOTE: a 0 or 1 value is assigned to each treatment pair.
3) This list is provided to the treatment administrator.

### 6.2.2.11.6.3. Removal of Animals from Study

Animals that are removed from the study after treatment with test article on Study Day 0 are not replaced.

### 6.2.2.12. Study Schedule

### Example First Cohort

Cohort 1 SD−20. ID, parity and calving date (gestational calf age) lists of eligible study subjects (Gestation Days 260-269) are obtained. Eligibility form/record for each subject is completed.

Dry Cows and Springing Heifers are randomized and assigned in Cohort 1 to Treatments (probiotic composition or control). Treatment administrator is informed of assignments.

Administration day—Sanitation supplies and probiotic composition applicator/tubes are prepared for Cohort 1 SD−14.

Probiotic composition is administered to animals in Cohort 1 and observations are recorded on the treatment administration evaluation form for each treated study subject.

Sanitation supplies and probiotic composition applicator are prepared for Cohort 1 SD−7 for the probiotic composition.

The probiotic composition is administered to animals in Cohort 1 SD−7 and observations on the treatment administration are recorded on the treatment evaluation form for each treated study subject

Calving—Calving record is completed (including calf weight and colostrum forms)

Recordation of daily milk production is started if farm has daily production measurement capability.

Daily Rectal Temperature and Vaginal Discharge Scoring days 1-7 for Cohort 1 is recorded.

Sanitation supplies and probiotic composition applicator for Cohort 1 SD+7 are prepared.

The probiotic composition is administered to animals in Cohort 1 SD+7 and observations on the treatment administration are recorded on the evaluation form for each treated study subject.

Sanitation supplies and applicator are prepared for Cohort 1 SD+14.

The probiotic composition is administered to animals in Cohort 1+SD14 and observations on the treatment administration are recorded on the evaluation form for each treated study subject in Cohort 1.

Health production and reproduction data are tracked for Cohort 1 to 120-150 DIM including any synchronization program treatments.

Any adverse events are monitored.

### 6.2.2.13. Study Procedures

### 6.2.2.13.1. Sanitation and Probiotic Composition Administration and Evaluation

For administration, the cow is sufficiently restrained (e.g., using feed bunk locking head gates).

For study subjects receiving the probiotic composition, the vulva is cleaned with water (e.g., using a spray bottle) to remove dirt, gentle scrubbing, if needed, with supplied peroxide or bleach wipes. The area is dried with a disposable towel.

The applicator tip is cleaned with provided wipes and dry with a clean paper towel. If needed, an appropriate obstetrical lubricant (J lube/OB Lube or similar) is applied to tip of the applicator. The probiotic composition itself can act as a lubricant.

The applicator tip is gently passed at a slight uphill angle past the vulva to deposit the gel between the cervix and mid vaginal vault.

To ensure a full-dose, the trigger of the applicator gun is slowly depressed while waiting for product to flow. Each full pull (2-clicks) of the trigger delivers 10 cc of product.

After administration, the applicator tip is slowly removed.

Details of each administration are recorded, including: Cow ID, date and time, site preparation, restraint used, dose delivered, and if the paste was retained. Any animal reaction is recorded, as well as the of ease of administration assessments:

Degree of restraint needed to apply the product using a Scale of 0-3, with 0 being use of a feed bunk head locks only and no other restraint. A Score of 1 is used for when the addition of moderate tail restraint is needed. A score of 2 indicates the need for significant tail restraint and a score of 3 indicates that additional restraint is required.

### Site Prep and Applicator Cleaned—0 for Yes, 1 for No

### Full Dose Received 0 for Yes, 1 for No

Gel Retention post dosing 0 for Yes, 1 for No
Immediate Cow Reaction Score 0 for none, 1 for some uneasiness and 2 for mild straining, 3 for significant straining and 4 if greater discomfort
Administration Evaluation 0—Easy no problems with insertion, administration, removal 1—Problem—detail in comments

Applicator is cleaned between uses using a peroxide based wipe and allowed to dry

Partially used cartridges are stored by removing applicator and placing tip back on cartridge. The used tube is stored in the same location as un-used tubes (cartridges). Herbie clips and EZ clips are saved, as they are needed for applying to other cartridges. Applicator tip and flexible PCV hose connector are not saved from week to week as the paste in the tube should not be used after storage for the week.

The cartridge has about 28 doses and is used to apply the probiotic composition to all study animals on a treatment day. For this study a new cartridge and applicator can be used on each treatment day.

Control animals have nothing administered during his administration phase, and are only noted in the treatment record as being present.

### 6.2.2.13.2. Calving Data Record

The following are recoded related to calving: Cow ID, date, initials of observer/recorder, Calving ease (score 1-4: 0 Normal no assistance needed, 1=calved with some difficulty but no assistance, 2=calving required minor assistance, 3=calving correction and then pulling, 4=calving required caesarean section or fetotomy), number of calves (single, twins etc), calf weight (if possible), and other observations (tares, problems). Dystocia is defined as any score >2. Cows that have dystocia induced trauma resulting in cervical or vaginal tears are excluded.

At the first milking post calving (within 4 hours) a milk colostrum sample is checked with either a hygrometer (Specific Gravity) or refractometer (Brix) when possible. The colostrometer is used with colostrum at room temperature (72° F.) when possible; when not possible, the temperature is recorded.

### 6.2.2.13.3. Post-Partum Health Definitions/Recording

The following are recorded when observed for all subjects:

Clinical hypocalcemia (milk fever)—Characterized by recumbency (inability to stand) and response to IV calcium solution. Occurs within 72 hours of calving.
Displaced Abomasum—Left or Right displacement of abomasum diagnosed by auscultation and percussion of the left or right abdomen between the 8th rib and the paralumbar fossa.
Retained Placenta—failure to expel fetal membranes within 24 hr. after parturition
Clinical ketosis-Elevated levels of ketones in blood, milk, or urine associated with anorexia and decreased rumen motility.
Clinical mastitis—Characterized by the presence of abnormal milk (clots, chunks, or thin-watery milk)
Pneumonia—Characterized by increased respiratory rate or effort, elevated rectal temperature, and a lack of signs of other clinical diseases.

### 6.2.2.13.1. Post-Partum Rectal Temp and Vaginal Discharge Evaluation (SD 0-14

Starting the morning following calving, all enrolled cows, both those who received the probiotic composition or control, have the following measured: rectal temperature and vaginal discharge. These are measured and recorded daily for at least 14 days (or until last administration of the probiotic composition, as the Cohort may have this last administration on a specific day beyond and not always on Day +14.

Rectal Temperature—The temperature is measured with GLA M900 thermometer or similar.

Vaginal Discharge—Scored at the time of obtaining rectal temperature—modified Williams system (0-clear mucus, 1-mucus containing flecks of pus, 2—discharge containing less than 50 percent pus, 3-discharge containing more than 50 percent pus) to classify vaginal mucus 4-50% pus and blood.

Recordings include Date, Cow ID, Vaginal Score, Rectal Temperature and initials of who made the assessment. The assessor scores if the APM (metritis) criteria are met (a vaginal discharge score of >2 and rectal temperature of >103.1° F.).

### 6.2.2.13.2. Vaginoscopy

Some study herds include vaginoscopy is their assessments. A vaginoscope is a clear plastic tube 1/½″ OD and about 21 inches long. The scope is cleaned using the provided hydrogen peroxide or bleach wipes and dried between use. This is conducted by the herd veterinarian or trained assessor.

The procedure is to moisten the speculum (scope) with 0.9% sodium chloride solution and insert the speculum into the vagina up to the outer cervical os. Cervix and vagina are visually examined for presence of pus and blood with the help of a flashlight. The amount of pus in the mucus is scored using a 4-point scoring system, as conducted for the discharge. The scope helps visualize the cervical os and deeper recess of the vagina. Herds that can conduct vaginoscopy conduct this procedure on the +7 and +14 post-partum before probiotic composition administration and then on the weekly SD 21 and 28, as this provides assessments of endometritis. Both probiotic composition and control animals are assessed. Recordings include Date, Cow ID, Vaginoscope Score and initials of who made the assessment.

### 6.2.2.13.3. Metritis Treatment

If the metritis criteria are met vaginal discharge score of >2 and rectal temperature of >103.1° F., the subject is treated with the farm's standard treatment and this is recorded in the health record. It is not mandatory that subjects that meet the criteria are treated, but subjects that do not meet the criteria are not treated.

### 6.2.2.13.4. Reproduction Data

To assess the impact of treatment with either the probiotic composition or control on reproduction endpoints the following are collected and recorded within herd electronic records or on paper:

Estrus dates if estrus observation is used on the farm;
Service (insemination) dates (used to calculate days to first insemination and number of inseminations);
Pregnancy assessments (used to calculate conception efficiency on each service and % non-return at various intervals (56 days 90 day) including Days Open and Calving Interval).
Other: Number culled for reproduction, Days in Herd, % Pregnant per insemination, all reproduction treatment used within and outside of the synchronization program

Each herd maintains the agreed on VWP and Synchronization program for the study duration.

### 6.2.2.13.5. Other Health Data

All other treatments, including vaccines, antibiotics, hormones, anti-inflammatories, infusions given by injection, intramammary, oral or intravaginal are recoded in each cow's health record. Records include, product name, dose, route, and date administered. This record is electronic or manual on paper.

### 6.2.2.13.6. Milk Production

Each herd has the ability to measure individual milk production, daily or a least every 14 days. Herds are on 2-3× milking. Milk components data is not collected. Data is captured electronically or manually.

### 6.2.2.14. Adverse Events

An adverse event (AE) is any observation in animals that is unfavorable and unintended and occurs after the use of the test product, whether or not considered to be product related.

Adverse events are classified as serious or non-serious. A serious AE is one that, in the opinion of the Study Director and in consultation with the Study Veterinarian, is life threatening, or causes death, persistent or significant disability/incapacity, severe lesions, or permanent or prolonged clinical signs. In addition, human exposure, anaphylactoid reactions as well as anticipated AEs that require medical attention over and above first aid measures are classified as serious.

Non-serious AEs are abnormal findings that do not fall into the description of serious AEs.

If an adverse event occurs that would be reasonably expected to compromise the integrity of the data, the animal is removed from the study.

If an adverse event occurs that would not be reasonably expected to compromise the integrity of the data, the animal is allowed to continue on the study and samples collected from an animal are analyzed.

### 6.2.3. Results

The probiotic composition is well tolerated in both dry/springing heifers and lactating cattle (cows and first calf heifers).

The probiotic composition has a positive impact on calving parameters, including calving ease, if assisted, fetal membrane retention, and colostrum quality/standard gravity (S.G.).

The probiotic composition has a positive impact on vaginal discharge scores during the first 2 weeks (0-14) post-partum and related progression/absence of uterine infections/retained placenta and metritis.

The probiotic composition has a positive impact on the use of antibiotics, infusions and hormones post-partum.

The probiotic composition has a positive impact on post-partum disorders including incidence of clinical hypocalcemia (milk fever), displaced abomasum, clinical ketosis, clinical mastitis, pneumonia and dystocia.

The probiotic composition has a positive impact on milk production through 120-150 days in milk (DIM).

The probiotic composition has a positive impact on key reproduction measures including: days to first insemination, % heats observed, days observed in heat, insemination rate (IR), conception rate (CR), first service, second service, third service, fourth service plus, pregnancy rate (PR), projected days open, % cows left herd, and % cows that left herd for reproduction.

### 7. SPECIFIC EMBODIMENTS

The present disclosure is exemplified by the specific embodiments below.

1. A probiotic composition in gel form suitable for intravaginal administration to a non-human animal, comprising:
(a) one or more strains of bacteria native to the vaginal tracts of healthy animals; and
(b) a non-aqueous base.

2. The probiotic composition of embodiment 1, wherein at least one of the one or more strains of bacteria is not native to the gastrointestinal tracts of healthy animals.

3. The probiotic composition of embodiment 1 or embodiment 2, wherein the one or more strains of bacteria comprise or consist of non-hemolytic, gram-positive, catalase-negative strains capable of growing under anaerobic conditions.

4. The probiotic composition of any one of embodiments 1 to 3, wherein the one or more strains of bacteria comprise or consist of one or more strains capable of reproducing at a pH in the range of 3 to 9.

5. The probiotic composition of embodiment 4, wherein the one or more strains of bacteria comprise or consist of one or more strains capable of reproducing at a pH in the range of 4 to 8.

6. The probiotic composition of embodiment 4, wherein the one or more strains of bacteria comprise or consist of one or more strains capable of reproducing at a pH in the range of 5 to 7.

7. The probiotic composition of any one of embodiments 1 to 6, wherein the one or more strains of bacteria comprise or consist of one or more strains capable of reproducing at a pH of 6 or higher.

8. The probiotic composition of any one of embodiments 1 to 7, wherein the one or more strains of bacteria comprise or consist of one or more strains capable of growing in a temperature range of 15° C. to 45° C.

9. The probiotic composition of embodiment 8, wherein the one or more strains of bacteria comprise or consist of one or more strains capable of growing at 20° C. and 39° C.

10. The probiotic composition of any one of embodiments 1 to 9, wherein the one or more strains of bacteria comprise or consist of one or more strains capable of auto-aggregation or co-aggregation.

11. The probiotic composition of any one of embodiments 1 to 10, wherein the one or more strains of bacteria comprise or consist of one or more strains that produce lactic acid.

12. The probiotic composition of any one of embodiments 1 to 11, wherein the one or more strains of bacteria comprise or consist of one or more strains that produce hydrogen peroxide.

13. The probiotic composition of any one of embodiments 1 to 12, wherein the one or more strains of bacteria comprise or consist of one or more strains capable of adhering to vaginal mucus.

14. The probiotic composition of any one of embodiments 1 to 13, wherein the one or more strains of bacteria comprise or consist of one or more strains that produce a bacteriocin.

15. The probiotic composition of any one of embodiments 1 to 14, wherein the one or more strains of bacteria comprise or consist of one or more strains of lactic acid bacteria (LAB).

16. The probiotic composition of embodiment 15, wherein the one or more strains of LAB comprise or consist of one or more strains of Abiotrophia, Aerococcus, Bifidobacterium, Carnobacterium, Enterococcus, Lactobacillus, Lactococcus, Leuconostoc, Oenococcus, Pediococcus, Streptococcus, Tetragenococcus, Vagococcus, Weissella, or a combination thereof.

17. The probiotic composition of embodiment 16, wherein the one or more strains of LAB comprise or consist of one or more strains of Lactobacillus.

18. The probiotic composition of embodiment 17, wherein the one or more strains of Lactobacillus comprise or consist of one or more strains of L. sakei, L. reuteri, L. rhamnosus, L. buchneri, L. mucosae, L. gasseri, L. delbrueckii, or a combination thereof.

19. The probiotic composition of embodiment 18, wherein the one or more strains of Lactobacillus comprise or consist of one or more strains of L. sakei.

20. The probiotic composition of embodiment 19, wherein the one or more strains of L. sakei comprise or consist of L. sakei FUA 3089.

21. The probiotic composition of any one of embodiments 16 to 20, wherein the one or more strains of LAB comprise or consist of one or more strains of Pediococcus.

22. The probiotic composition of embodiment 21, wherein the one or more strains of Pediococcus comprise or consist of one or more strains of P. acidilactici.

23. The probiotic composition of embodiment 22, wherein the one or more strains of P. acidilactici comprise or consist of P. acidilactici FUA 3138 and/or P. acidilactici FUA 3140.

24. The probiotic composition of embodiment 23, wherein the one or more strains of P. acidilactici comprise or consist of P. acidilactici FUA 3138 and P.s acidilactici FUA 3140.

25. The probiotic composition of any one of embodiments 1 to 24, which comprises two or more strains of bacteria.

26. The probiotic composition of embodiment 25, which comprises or consists of two strains of bacteria.

27. The probiotic composition of embodiment 26, wherein each of the two strains accounts for 10% to 90% of the total amount of the bacteria in the probiotic composition on a CFU basis, provided that the amounts of the two strains are selected so that the sum of the amounts of the two strains does not exceed 100%.

28. The probiotic composition of embodiment 27, wherein one of the two strains accounts for 10% to 20% of the total amount of bacteria in the probiotic composition on a CFU basis.

29. The probiotic composition of embodiment 27, wherein one of the two strains accounts for 10% to 30% of the total amount of bacteria in the probiotic composition on a CFU basis.

30. The probiotic composition of embodiment 27, wherein one of the two strains accounts for 10% to 40% of the total amount of bacteria in the probiotic composition on a CFU basis.

31. The probiotic composition of embodiment 27, wherein one of the two strains accounts for 10% to 50% of the total amount of bacteria in the probiotic composition on a CFU basis.

32. The probiotic composition of embodiment 27, wherein one of the two strains accounts for 10% to 60% of the total amount of bacteria in the probiotic composition on a CFU basis.

33. The probiotic composition of embodiment 27, wherein one of the two strains accounts for 10% to 70% of the total amount of bacteria in the probiotic composition on a CFU basis.

34. The probiotic composition of embodiment 27, wherein one of the two strains accounts for 10% to 80% of the total amount of bacteria in the probiotic composition on a CFU basis.

35. The probiotic composition of embodiment 27, wherein one of the two strains accounts for 20% to 30% of the total amount of bacteria in the probiotic composition on a CFU basis.

36. The probiotic composition of embodiment 27, wherein one of the two strains accounts for 20% to 40% of the total amount of bacteria in the probiotic composition on a CFU basis.

37. The probiotic composition of embodiment 27, wherein one of the two strains accounts for 20% to 50% of the total amount of bacteria in the probiotic composition on a CFU basis.

38. The probiotic composition of embodiment 27, wherein one of the two strains accounts for 20% to 60% of the total amount of bacteria in the probiotic composition on a CFU basis.

39. The probiotic composition of embodiment 27, wherein one of the two strains accounts for 20% to 70% of the total amount of bacteria in the probiotic composition on a CFU basis.

40. The probiotic composition of embodiment 27, wherein one of the two strains accounts for 20% to 80% of the total amount of bacteria in the probiotic composition on a CFU basis.

41. The probiotic composition of embodiment 27, wherein one of the two strains accounts for 20% to 90% of the total amount of bacteria in the probiotic composition on a CFU basis.

42. The probiotic composition of embodiment 27, wherein one of the two strains accounts for 30% to 40% of the total amount of bacteria in the probiotic composition on a CFU basis.

43. The probiotic composition of embodiment 27, wherein one of the two strains accounts for 30% to 50% of the total amount of bacteria in the probiotic composition on a CFU basis.

44. The probiotic composition of embodiment 27, wherein one of the two strains accounts for 30% to 60% of the total amount of bacteria in the probiotic composition on a CFU basis.

45. The probiotic composition of embodiment 27, wherein one of the two strains accounts for 30% to 70% of the total amount of bacteria in the probiotic composition on a CFU basis.

46. The probiotic composition of embodiment 27, wherein one of the two strains accounts for 30% to 80% of the total amount of bacteria in the probiotic composition on a CFU basis.

47. The probiotic composition of embodiment 27, wherein one of the two strains accounts for 30% to 90% of the total amount of bacteria in the probiotic composition on a CFU basis.

48. The probiotic composition of embodiment 27, wherein one of the two strains accounts for 40% to 50% of the total amount of bacteria in the probiotic composition on a CFU basis.

49. The probiotic composition of any one of embodiments 1 to 24, which comprises or consists of three or more strains of bacteria.

50. The probiotic composition of embodiment 49, which comprises or consists of three strains of bacteria.

51. The probiotic composition of embodiment 50, wherein each of the three strains accounts for 10% to 50% of the total amount of bacteria in the probiotic composition on a CFU basis, provided that the amounts of the three strains are selected so that the sum of the amounts of the three strains does not exceed 100%.

52. The probiotic composition of embodiment 51, wherein each of the three strains is at least 5% of the total amount of bacteria in the probiotic composition on a CFU basis.

53. The probiotic composition of embodiment 51, wherein each of the three strains is at least 10% of the total amount of bacteria in the probiotic composition on a CFU basis.

54. The probiotic composition of embodiment 51, wherein each of the three strains is at least 20% of the total amount of bacteria in the probiotic composition on a CFU basis.

55. The probiotic composition of embodiment 51, wherein each of the three strains is at least 25% of the total amount of bacteria in the probiotic composition on a CFU basis.

56. The probiotic composition of any one of embodiments 1 to 24 or 49 to 51, which comprises or consists of L. sakei FUA 3089, P. acidilactici FUA 3138 and P. acidilactici FUA 3140.

57. The probiotic composition of any one of embodiments 1 to 56, which is free of contaminating bacteria.

58. The probiotic composition of any one of embodiments 1 to 57, wherein the one or more strains of bacteria are dried.

59. The probiotic composition of any one of embodiments 1 to 58, wherein the one or more strains of bacteria are lyophilized.

60. The probiotic composition of any one of embodiments 1 to 58, wherein the one or more strains of bacteria are spray-dried.

61. The probiotic composition of any one of embodiments 1 to 60, wherein the probiotic composition comprises 10³ to 10¹⁰ total colony forming units (CFU) per 1 ml.

62. The probiotic composition of embodiment 61, wherein the probiotic composition comprises 10³ to 10¹⁰ total CFU per 1 ml.

63. The probiotic composition of embodiment 61, wherein the probiotic composition comprises 10³ to 10⁹ total CFU per 1 ml.

64. The probiotic composition of embodiment 61, wherein the probiotic composition comprises 10³ to 10⁸ total CFU per 1 ml.

65. The probiotic composition of embodiment 61, wherein the probiotic composition comprises 10³ to 10⁷ total CFU per 1 ml.

66. The probiotic composition of embodiment 61, wherein the probiotic composition comprises 10³ to 10⁶ total CFU per 1 ml.

67. The probiotic composition of embodiment 61, wherein the probiotic composition comprises 10³ to 10⁵ total CFU per 1 ml.

68. The probiotic composition of embodiment 61, wherein the probiotic composition comprises 10³ to 10⁴ total CFU per 1 ml.

69. The probiotic composition of embodiment 61, wherein the probiotic composition comprises 10⁴ to 10¹⁰ total CFU per 1 ml.

70. The probiotic composition of embodiment 61, wherein the probiotic composition comprises 10⁴ to 10⁹ total CFU per 1 ml.

71. The probiotic composition of embodiment 61, wherein the probiotic composition comprises 10⁴ to 10⁸ total CFU per 1 ml.

72. The probiotic composition of embodiment 61, wherein the probiotic composition comprises 10⁴ to 10⁷ total CFU per 1 ml.

73. The probiotic composition of embodiment 61, wherein the probiotic composition comprises 10⁴ to 10⁶ total CFU per 1 ml.

74. The probiotic composition of embodiment 61, wherein the probiotic composition comprises 10⁴ to 10⁵ total CFU per 1 ml.

75. The probiotic composition of embodiment 61, wherein the probiotic composition comprises 10⁵ to 10¹⁰ total CFU per 1 ml.

76. The probiotic composition of embodiment 61, wherein the probiotic composition comprises 10⁵ to 10⁹ total CFU per 1 ml.

77. The probiotic composition of embodiment 61, wherein the probiotic composition comprises 10⁵ to 10⁸ total CFU per 1 ml.

78. The probiotic composition of embodiment 61, wherein the probiotic composition comprises 10⁵ to 10⁷ total CFU per 1 ml.

79. The probiotic composition of embodiment 61, wherein the probiotic composition comprises 10⁵ to 10⁶ total CFU per 1 ml.

80. The probiotic composition of embodiment 61, wherein the probiotic composition comprises 10⁶ to 10¹⁰ total CFU per 1 ml.

81. The probiotic composition of embodiment 61, wherein the probiotic composition comprises 10⁶ to 10⁹ total CFU per 1 ml.

82. The probiotic composition of embodiment 61, wherein the probiotic composition comprises 10⁶ to 10⁸ total CFU per 1 ml.

83. The probiotic composition of embodiment 61, wherein the probiotic composition comprises 10⁶ to 10⁷ total CFU per 1 ml.

84. The probiotic composition of embodiment 61, wherein the probiotic composition comprises 10⁷ to 10¹⁰ total CFU per 1 ml.

85. The probiotic composition of embodiment 61, wherein the probiotic composition comprises 10⁷ to 10⁹ total CFU per 1 ml.

86. The probiotic composition of embodiment 61, wherein the probiotic composition comprises 10⁷ to 10⁸ total CFU per 1 ml.

87. The probiotic composition of embodiment 61, wherein the probiotic composition comprises 10⁸ to 10¹⁰ total CFU per 1 ml.

88. The probiotic composition of embodiment 61, wherein the probiotic composition comprises 10⁸ to 10⁹ total CFU per 1 ml.

89. The probiotic composition of embodiment 61, wherein the probiotic composition comprises 10⁹ to 10¹⁰ total CFU per 1 ml.

90. The probiotic composition of embodiment 61, wherein the probiotic composition comprises 0.2 billion to 0.8 billion total CFU per 1 ml.

91. The probiotic composition of embodiment 61, wherein the probiotic composition comprises 0.2 billion to 0.6 billion total CFU per 1 ml.

92. The probiotic composition of embodiment 61, wherein the probiotic composition comprises 0.4 billion to 1 billion total CFU per 1 ml.

93. The probiotic composition of embodiment 61, wherein the probiotic composition comprises 0.4 billion to 0.8 billion total CFU per 1 ml.

94. The probiotic composition of embodiment 61, wherein the probiotic composition comprises 0.4 billion to 0.6 billion total CFU per 1 ml.

95. The probiotic composition of embodiment 61, wherein the probiotic composition comprises 0.6 billion to 1 billion total CFU per 1 ml.

96. The probiotic composition of embodiment 61, wherein the probiotic composition comprises 0.6 billion to 0.8 billion total CFU per 1 ml.

97. The probiotic composition of embodiment 61, wherein the probiotic composition comprises 0.8 billion to 1 billion total CFU per 1 ml.

98. The probiotic composition of any one of embodiments 1 to 97, wherein the non-aqueous base comprises one or more oils.

99. The probiotic composition of embodiment 98, wherein the one or more oils comprise or consist of one or more plant-derived oils.

100. The probiotic composition of embodiment 99, wherein the plant-derived oils comprise or consist of one or more non-GMO plant-derived oils.

101. The probiotic composition of embodiment 99 or embodiment 100, wherein the one or more plant-derived oils comprise or consist of soybean oil, borage seed oil, flaxseed oil, evening primrose oil, canola oil, safflower oil, sunflower oil, grapeseed oil, sesame oil, hemp seed oil, pumpkin seed oil, or a combination thereof.

102. The probiotic composition of embodiment 99 or embodiment 100, wherein the one or more plant-derived oils comprise or consist of soybean oil.

103. The probiotic composition of embodiment 102, wherein the soybean oil is non-GMO soybean oil.

104. The probiotic composition of any one of embodiments 1 to 103, wherein the non-aqueous base further comprises or further comprises one or more waxes.

105. The probiotic composition of embodiment 104, wherein the one or more waxes comprise or consist of beeswax.

106. The probiotic composition of any one of embodiments 1 to 105, wherein the non-aqueous base comprises or further comprises one or more fatty substances.

107. The probiotic composition of embodiment 106, wherein the one or more fatty substances comprise or consist of cocoa butter, a cocoa butter substitute, or a combination thereof.

108. The probiotic composition of embodiment 107, wherein the one or more fatty substances comprise or consist of cocoa butter.

109. The probiotic composition of embodiment 107 or embodiment 108, wherein the one or more fatty substances comprise or consist of a cocoa butter substitute.

110. The probiotic composition of any one of embodiments 107 to 109, wherein the cocoa butter substitute comprises or consists of synthetic triglycerides, triglycerides from one or more plant oils, or a combination thereof.

111. The probiotic composition of embodiment 110, wherein the cocoa butter substitute comprises or consists of synthetic triglycerides.

112. The probiotic composition of embodiment 110 or embodiment 111, wherein the cocoa butter substitute comprises or consists of triglycerides from one or more plant oils.

113. The probiotic composition of embodiment 112, wherein the triglycerides from one or more plant oils comprise or consist of triglycerides from palm oil, palm kernel oil, coconut oil, or a combination thereof.

114. The probiotic composition of any one of embodiments 1 to 113, wherein the non-aqueous base comprises or further comprises glycerinated gelatin.

115. The probiotic composition of any one of embodiments 1 to 114, wherein the non-aqueous base comprises or further comprises one or more hydrophilic polymers.

116. The probiotic composition of embodiment 115, wherein the hydrophilic polymers comprise or consist of one or more polyethylene glycols (PEGs).

117. The probiotic composition of embodiment 116, wherein the hydrophilic polymers comprise or consist of a combination of PEGs of different molecular weight.

118. The probiotic composition of any one of embodiments 98 to 117, wherein the non-aqueous base further comprises one or more thickeners.

119. The probiotic composition of embodiment 118, wherein the one or more thickeners comprise or consist of silicon dioxide, calcium sulfate, sodium sulfate, magnesium sulfate, one or more oligosaccharides, one or more polysaccharides, one or more emulsifiers, one or more bentonite clays, sodium alginate, whey protein, or a combination thereof.

120. The probiotic composition of embodiment 119, wherein the one or more thickeners comprise or consist of silicon dioxide.

121. The probiotic composition of embodiment 119 or embodiment 120, wherein the one or more thickeners comprise or consist of one or more polysaccharides.

122. The probiotic composition of embodiment 121, wherein the one or more polysaccharides comprise or consist of one or more starches, dextrins, maltodextrins, or a combination thereof.

123. The probiotic composition of embodiment 121, wherein the one or more polysaccharides comprise or consist of one or more starches, dextrins, maltodextrins, pullulan, pullulan derivatives, agarose or a combination thereof.

124. The probiotic composition of embodiment 123, wherein the one or more polysaccharides comprise or consist or pullulan.

125. The probiotic composition of embodiment 123 or embodiment 124, wherein the one or more polysaccharides comprise or consist of one or more pullulan derivatives.

126. The probiotic composition of embodiment 125, wherein the one or more pullulan derivatives comprise or consist of esterified pullulan, etherified pullulan, hydrogenated pullulan, sulfated pullulan, chlorinated pullulan, cholesterol substituted pullulan, fatty acid substituted pullulan, or a combination thereof.

127. The probiotic composition of embodiment 126, wherein the one or more pullulan derivatives comprise or consist of esterified pullulan.

128. The probiotic composition of embodiment 126 of embodiment 127, wherein the one or more pullulan derivatives comprise or consist of etherified pullulan.

129. The probiotic composition of any one of embodiments 126 to 128, wherein the one or more pullulan derivatives comprise or consist of hydrogenated pullulan.

130. The probiotic composition of any one of embodiments 126 to 129, wherein the one or more pullulan derivatives comprise or consist of sulfated pullulan.

131. The probiotic composition of any one of embodiments 126 to 130, wherein the one or more pullulan derivatives comprise or consist of chlorinated pullulan.

132. The probiotic composition of any one of embodiments 126 to 131, wherein the one or more pullulan derivatives comprise or consist of cholesterol substituted pullulan.

133. The probiotic composition of any one of embodiments 126 to 132, wherein the one or more pullulan derivatives comprise or consist of fatty acid substituted pullulan.

134. The probiotic composition of any one of embodiments 123 to 133, wherein the one or more polysaccharides comprise or consist of agarose.

135. The probiotic composition of any one of embodiments 122 to 134, wherein the one or more polysaccharides comprise or consist of one or more starches.

136. The probiotic composition of embodiment 135, wherein the one or more starches comprise or consist of corn starch, potato starch, wheat starch, oat starch, barley starch, rice starch, sorghum starch, a legume starch (e.g., from a pea or a bean), tapioca, or a combination thereof.

137. The probiotic composition of embodiment 136, wherein the one or more starches comprise or consist of corn starch.

138. The probiotic composition of any one of embodiments 135 to 137, wherein the one or more starches comprise or consist of native starches, modified starches, or a combination thereof.

139. The probiotic composition of embodiment 138, wherein the one or more starches comprise or consist of one or more native starches.

140. The probiotic composition of embodiment 138 or embodiment 139, wherein the one or more starches comprise or consist of one or more modified starches.

141. The probiotic composition of embodiment 140, wherein the one or more modified starches comprise or consist of one or more chemically treated starches, one or more alkali and/or acid washed starches, one or more enzymatically hydrolyzed starches, one or more bleached starches, one or more esterified starches, one or more cross-linked starches, one or more ionized starches, one or more oxidized starches, or a combination thereof.

142. The probiotic composition of any one of embodiments 118 to 141, wherein the one or more thickeners comprise or consist of corn starch and silicon dioxide.

143. The probiotic composition of any one of embodiments 118 to 142, wherein the one or more thickeners comprise or consist of one or more emulsifiers.

144. The probiotic composition of embodiment 143, wherein the one or more emulsifiers comprise or consist of one or more lecithins.

145. The probiotic composition of any one of embodiments 98 to 144, which further comprises one or more prebiotics.

146. The probiotic composition of embodiment 145, wherein the one or more prebiotics comprise or consist of one or more monosaccharides, one or more disaccharides, one or more oligosaccharides, one or more polysaccharides, a fermentation product, or a combination thereof.

147. The probiotic composition of embodiment 146, wherein the one or more prebiotics comprise or consist of one or more monosaccharides.

148. The probiotic composition of embodiment 147, wherein the one or more prebiotics comprise or consist of dextrose.

149. The probiotic of embodiment 148 wherein the dextrose is anhydrous dextrose.

150. The probiotic composition of any one of embodiments 145 to 149, wherein the one or more prebiotics comprise or consist of one or more disaccharides.

151. The probiotic composition of embodiment 150, wherein the one or more disaccharides comprise or consist of sucrose.

152. The probiotic composition of embodiment 151, wherein the sucrose is powdered sugar.

153. The probiotic composition of any one of embodiments 150 to 152, wherein the one or more disaccharides comprise or consist of trehalose.

154. The probiotic composition of any one of embodiments 145 to 153, wherein the one or more prebiotics comprise or consist of one or more oligosaccharides.

155. The probiotic composition of embodiment 154, wherein the one or more oligosaccharides comprise or consist of one or more fructooligosaccharides (FOS).

156. The probiotic composition of any one of embodiments 145 to 155, wherein the one or more prebiotics comprise or consist of one or more fermentation products.

157. The probiotic composition of embodiment 156, wherein the one or more fermentation products comprise or consist of one or more fermentation products obtained or obtainable by a process comprising:
(a) separating biomass from a fermentation broth to produce a depleted fermentation broth; and
(b) drying the depleted fermentation broth produced in step (a) to produce a fermentation product.

158. The probiotic composition of embodiment 157, wherein the process further comprises combining one or more reagents with the depleted fermentation broth prior to step (b).

159. The probiotic composition of embodiment 158, wherein the one or more reagents comprise one or more thickeners.

160. The probiotic composition of embodiment 159, wherein the one or more thickeners comprise maltodextrin.

161. The probiotic composition of any one of embodiments 157 to 160, wherein the biomass is separated from the fermentation broth by centrifugation.

162. The probiotic product of any one of embodiments 157 to 161, wherein step (b) comprises spray-drying.

163. The probiotic product of any one of embodiments 156 to 162, wherein the fermentation product comprises one or more bacteriocins.

164. The probiotic product of embodiment 163, wherein the one or more bacteriocins comprise pediocin AcH/PA-1.

165. The probiotic composition of any one of embodiments 156 to 164, wherein the one or more fermentation products comprise or consist of one or more fermentation products from one or more of the strains of bacteria in the probiotic composition.

166. The probiotic composition of any one of embodiments 156 to 165, wherein the one or more fermentation products comprise or consist of a Lactobacillus sakei fermentation product and/or a Pediococcus acidilactici fermentation product.

167. The probiotic composition of embodiment 166, wherein the one or more fermentation products comprise or consist of a Lactobacillus sakei fermentation product and a Pediococcus acidilactici fermentation product.

168. The probiotic composition of any one of embodiments 156 to 167, wherein the one or more fermentation products comprise or consist of spray-dried fermentation products.

169. The probiotic composition of any one of embodiments 1 to 168, which further comprises one or more carotenoids.

170. The probiotic composition of embodiment 169, wherein the one or more carotenoids comprise or consist of beta-carotene.

171. The probiotic composition of any one of embodiments 1 to 170, wherein the probiotic composition has a National Lubricating Grease Institute (NLGI) consistency grade of 000 to 6.

172. The probiotic composition of embodiment 171, wherein the probiotic composition has a NLGI consistency grade of 000 to 5.

173. The probiotic composition of embodiment 171, wherein the probiotic composition has a NLGI consistency grade of 000 to 4.

174. The probiotic composition of embodiment 171, wherein the probiotic composition has a NLGI consistency grade of 000 to 3.

175. The probiotic composition of embodiment 171, wherein the probiotic composition has a NLGI consistency grade of 000 to 2.

176. The probiotic composition of embodiment 171, wherein the probiotic composition has a NLGI consistency grade of 000 to 1.

177. The probiotic composition of embodiment 171, wherein the probiotic composition has a NLGI consistency grade of 000 to 0.

178. The probiotic composition of embodiment 171, wherein the probiotic composition has a NLGI consistency grade of 000 to 00.

179. The probiotic composition of embodiment 171, wherein the probiotic composition has a NLGI consistency grade of 00 to 6.

180. The probiotic composition of embodiment 171, wherein the probiotic composition has a NLGI consistency grade of 00 to 5.

181. The probiotic composition of embodiment 171, wherein the probiotic composition has a NLGI consistency grade of 00 to 4.

182. The probiotic composition of embodiment 171, wherein the probiotic composition has a NLGI consistency grade of 00 to 3.

183. The probiotic composition of embodiment 171, wherein the probiotic composition has a NLGI consistency grade of 00 to 2.

184. The probiotic composition of embodiment 171, wherein the probiotic composition has a NLGI consistency grade of 00 to 1.

185. The probiotic composition of embodiment 171, wherein the probiotic composition has a NLGI consistency grade of 00 to 0.

186. The probiotic composition of embodiment 171, wherein the probiotic composition has a NLGI consistency grade of 0 to 6.

187. The probiotic composition of embodiment 171, wherein the probiotic composition has a NLGI consistency grade of 0 to 5.

188. The probiotic composition of embodiment 171, wherein the probiotic composition has a NLGI consistency grade of 0 to 4.

189. The probiotic composition of embodiment 171, wherein the probiotic composition has a NLGI consistency grade of 0 to 3.

190. The probiotic composition of embodiment 171, wherein the probiotic composition has a NLGI consistency grade of 0 to 2.

191. The probiotic composition of embodiment 171, wherein the probiotic composition has a NLGI consistency grade of 0 to 1.

192. The probiotic composition of embodiment 171, wherein the probiotic composition has a NLGI consistency grade of 1 to 6.

193. The probiotic composition of embodiment 171, wherein the probiotic composition has a NLGI consistency grade of 1 to 5.

194. The probiotic composition of embodiment 171, wherein the probiotic composition has a NLGI consistency grade of 1 to 4.

195. The probiotic composition of embodiment 171, wherein the probiotic composition has a NLGI consistency grade of 1 to 3.

196. The probiotic composition of embodiment 171, wherein the probiotic composition has a NLGI consistency grade of 1 to 2.

197. The probiotic composition of embodiment 171, wherein the probiotic composition has a NLGI consistency grade of 2 to 6.

198. The probiotic composition of embodiment 171, wherein the probiotic composition has a NLGI consistency grade of 2 to 5.

199. The probiotic composition of embodiment 171, wherein the probiotic composition has a NLGI consistency grade of 2 to 4.

200. The probiotic composition of embodiment 171, wherein the probiotic composition has a NLGI consistency grade of 2 to 3.

201. The probiotic composition of embodiment 171, wherein the probiotic composition has a NLGI consistency grade of 3 to 6.

202. The probiotic composition of embodiment 171, wherein the probiotic composition has a NLGI consistency grade of 3 to 5.

203. The probiotic composition of embodiment 171, wherein the probiotic composition has a NLGI consistency grade of 3 to 4.

204. The probiotic composition of embodiment 171, wherein the probiotic composition has a NLGI consistency grade of 4 to 6.

205. The probiotic composition of embodiment 171, wherein the probiotic composition has a NLGI consistency grade of 4 to 5.

206. The probiotic composition of embodiment 171, wherein the probiotic composition has a NLGI consistency grade of 5 to 6.

207. The probiotic composition of embodiment 171, wherein the probiotic composition has a NLGI consistency grade of 000.

208. The probiotic composition of embodiment 171, wherein the probiotic composition has a NLGI consistency grade of 00.

209. The probiotic composition of embodiment 171, wherein the probiotic composition has a NLGI consistency grade of 0.

210. The probiotic composition of embodiment 171, wherein the probiotic composition has a NLGI consistency grade of 1.

211. The probiotic composition of embodiment 171, wherein the probiotic composition has a NLGI consistency grade of 2.

212. The probiotic composition of embodiment 171, wherein the probiotic composition has a NLGI consistency grade of 3.

213. The probiotic composition of embodiment 171, wherein the probiotic composition has a NLGI consistency grade of 4.

214. The probiotic composition of embodiment 171, wherein the probiotic composition has a NLGI consistency grade of 5.

215. The probiotic composition of embodiment 171, wherein the probiotic composition has a NLGI consistency grade of 6.

216. The probiotic composition of any one of embodiments 171 to 215, wherein the NLGI consistency grade is the NGLI consistency grade as measured by ASTM D937-07 on an unworked sample of the probiotic composition.

217. The probiotic composition of any one of embodiments 171 to 215, wherein the NLGI consistency grade is the NGLI consistency grade as measured by ASTM D 217-02 on an unworked sample of the probiotic composition.

218. The probiotic composition of any one of embodiments 1 to 170, wherein the probiotic composition has a viscosity at 20° C. between 30,000 cP and 1 M cP.

219. The probiotic composition of embodiment 218, wherein the probiotic composition has a viscosity at 20° C. between 30,000 cP and 500,000 cP.

220. The probiotic composition of embodiment 218, wherein the probiotic composition has a viscosity at 20° C. between 30,000 cP and 250,000 cP.

221. The probiotic composition of embodiment 218, wherein the probiotic composition has a viscosity at 20° C. between 30,000 cP and 100,000 cP.

222. The probiotic composition of embodiment 218, wherein the probiotic composition has a viscosity at 20° C. between 50,000 cP and 500,000 cP.

223. The probiotic composition of embodiment 218, wherein the probiotic composition has a viscosity at 20° C. between 50,000 cP and 250,000 cP.

224. The probiotic composition of embodiment 218, wherein the probiotic composition has a viscosity at 20° C. between 50,000 cP and 100,000 cP.

225. The probiotic composition of embodiment 218, wherein the probiotic composition has a viscosity at 20° C. between 75,000 cP and 500,000 cP.

226. The probiotic composition of embodiment 218, wherein the probiotic composition has a viscosity at 20° C. between 75,000 cP and 250,000 cP.

227. The probiotic composition of embodiment 218, wherein the probiotic composition has a viscosity at 20° C. between 75,000 cP and 100,000 cP.

228. The probiotic composition of embodiment 218, wherein the probiotic composition has a viscosity at 20° C. between 100,000 cP and 500,000 M cP.

229. The probiotic composition of embodiment 218, wherein the probiotic composition has a viscosity at 20° C. between 100,000 cP and 250,000 M cP.

230. The probiotic composition of any one of embodiments 1 to 229, wherein the probiotic composition has a bioadhesive force ranging from 5,000 to 20,000 dyne/cm² as measured by the in vitro bioadhesion assay described in El-Kamel and El-Khatib, 2006, Drug Delivery, 13(2):143-148.

231. The probiotic composition of any one of embodiments 1 to 230, wherein the probiotic composition has a specific gravity ranging from 1.0 to 1.2.

232. The probiotic composition of embodiment 231, wherein the probiotic composition has a specific gravity ranging from 1.1 to 1.2.

233. The probiotic composition of any one of embodiments 1 to 232, wherein the probiotic composition is not runny at temperatures ranging from 10° C. to 70° C.

234. The probiotic composition of any one of embodiments 1 to 232, wherein the probiotic composition is not runny at temperatures ranging from 10° C. to 60° C.

235. The probiotic composition of any one of embodiments 1 to 232, wherein the probiotic composition is not runny at temperatures ranging from 10° C. to 50° C.

236. The probiotic composition of any one of embodiments 1 to 232, wherein the probiotic composition is not runny at temperatures ranging from 10° C. to 40° C.

237. The probiotic composition of any one of embodiments 1 to 236, which has a water content of less than 5% by weight.

238. The probiotic composition of any one of embodiments 1 to 236, which has a water content of less than 4% by weight.

239. The probiotic composition of any one of embodiments 1 to 236 which has a water content of less than 3% by weight.

240. The probiotic composition of any one of embodiments 1 to 236, which has a water content of less than 2% by weight.

241. The probiotic composition of any one of embodiments 1 to 236, which has a water content of less than 1% by weight.

242. The probiotic composition of any one of embodiments 237 to 241, which has a water content of at least 0.01% by weight.

243. The probiotic composition of embodiment 242, which has a water content of at least 0.1% by weight.

244. The probiotic composition of any one of embodiments 1 to 241, which comprises:
(a) the one or more strains of bacteria;
(b) dextrose, which is optionally anhydrous dextrose;
(c) sucrose, which is optionally powdered sugar;
(d) corn starch;
(e) fructooligosaccharide;
(f) silicon dioxide;
(g) soybean oil, which is optionally non-GMO soybean oil; and
(h) a Lactobacillus sakei fermentation product and a Pediococcus acidilactici fermentation product.

245. The probiotic composition of embodiment 244, which comprises:
(a) the one or more strains of bacteria;
(b) 2% to 6% dextrose, by weight of the composition;
(c) 3% to 9% sucrose, by weight of the composition;
(d) 2% to 5% corn starch, by weight of the composition;
(e) 4% to 12% fructooligosaccharide, by weight of the composition;
(f) 10% to 20% silicon dioxide, by weight of the composition;
(g) 50% to 70% soybean oil, by weight of the composition; and
(h) a Lactobacillus sakei fermentation product and a Pediococcus acidilactici fermentation product, which together are 0.5% to 3% of the composition by weight,

provided that the amounts of components (a)-(h) are selected so that the sum of the weights of the components does not exceed 100% of the weight of the probiotic composition.

246. The probiotic composition of embodiment 245, wherein the sum of the weights of components (a)-(h) is 100% of the weight of the probiotic composition.

247. The probiotic composition of embodiment 245 or embodiment 246, which comprises:
(a) the one or more strains of bacteria;
(b) about 3.2% to about 4.8% dextrose, by weight of the composition;
(c) about 4.8% to about 7.2% sucrose, by weight of the composition;
(d) about 2.8% to about 4.2% corn starch, by weight of the composition;
(e) about 6.4% to about 9.6% fructooligosaccharide, by weight of the composition;
(f) about 11.2% to about 16.8% silicon dioxide, by weight of the composition;
(g) 50% to about 70% soybean oil, by weight of the composition; and
(h) a Lactobacillus sakei fermentation product and a Pediococcus acidilactici fermentation product, which together are about 0.8% to about 1.2% of the composition by weight.

248. The probiotic composition of embodiment 245 or embodiment 246, which comprises:
(a) the one or more strains of bacteria;
(b) about 4% dextrose, by weight of the composition;
(c) about 6% sucrose, by weight of the composition;
(d) about 3.5% corn starch, by weight of the composition;
(e) about 8% fructooligosaccharide, by weight of the composition;
(f) about 14% silicon dioxide, by weight of the composition;
(g) about 63% soybean oil, by weight of the composition; and
(h) a Lactobacillus sakei fermentation product and a Pediococcus acidilactici fermentation product, which together are about 1% of the composition by weight.

249. The probiotic composition of any one of embodiments 244 to 248, wherein the dextrose is anhydrous dextrose.

250. The probiotic composition of any one of embodiments 244 to 249, wherein the sucrose is powdered sugar.

251. The probiotic composition of any one of embodiments 244 to 250, wherein the soybean oil is non-GMO soybean oil.

252. The probiotic composition of any one of embodiments 1 to 241, which comprises:
(a) the one or more strains of bacteria;
(b) sucrose, which is optionally powdered sugar;
(c) corn starch;
(d) fructooligosaccharide;
(e) silicon dioxide; and
(f) soybean oil, which is optionally non-GMO soybean oil.

253. The probiotic composition of embodiment 252, which comprises:
(a) the one or more strains of bacteria;
(b) 3% to 9% sucrose, by weight of the composition;
(c) 2% to 8% corn starch, by weight of the composition;
(d) 4% to 12% fructooligosaccharide, by weight of the composition;
(e) 10% to 20% silicon dioxide, by weight of the composition; and
(f) 50% to 70% soybean oil, by weight of the composition;
provided that the amounts of components (a)-(f) are selected so that the sum of the weights of the components does not exceed 100% of the weight of the probiotic composition.

254. The probiotic composition of embodiment 253, wherein the sum of the weights of components (a)-(f) is 100% of the weight of the probiotic composition.

255. The probiotic composition of embodiment 253 or embodiment 254, which comprises:
(a) the one or more strains of bacteria;
(b) about 6.3% sucrose, by weight of the composition;
(c) about 4.7% corn starch, by weight of the composition;
(d) about 8% fructooligosaccharide, by weight of the composition;
(e) about 15% silicon dioxide, by weight of the composition; and
(f) about 66% soybean oil, by weight of the composition.

256. The probiotic composition of embodiment 253, which comprises:
(a) the one or more strains of bacteria;
(b) one or more fermentation products;
(c) about 6.3% sucrose, by weight of the composition;
(d) about 3.6% corn starch, by weight of the composition;
(e) about 8% fructooligosaccharide, by weight of the composition;
(f) about 15% silicon dioxide, by weight of the composition; and
(g) about 66% soybean oil, by weight of the composition.

257. The probiotic composition of embodiment 256, wherein the sum of the weights of components (a)-(g) is 100% of the weight of the probiotic composition.

258. The probiotic composition of any one of embodiments 252 to 257, wherein the sucrose is powdered sugar.

259. The probiotic composition of any one of embodiments 252 to 258, wherein the soybean oil is non-GMO soybean oil.

260. The probiotic composition of any one of embodiments 1 to 259, which is free of animal protein.

261. The probiotic composition of any one of embodiments 1 to 260, which is free of any components produced by a genetically modified organism.

262. The probiotic composition of any one of embodiments 1 to 261, wherein the probiotic composition maintains at least 60% of its CFU after 3 months of storage at 20° C.

263. The probiotic composition of any one of embodiments 1 to 261, wherein the probiotic composition maintains at least 80% of its CFU after 3 months of storage at 20° C.

264. The probiotic composition of any one of embodiments 1 to 261, wherein the probiotic composition maintains at least 60% of its CFU after 6 months of storage at 20° C.

265. The probiotic composition of any one of embodiments 1 to 261, wherein the probiotic composition maintains at least 80% of its CFU after 6 months of storage at 20° C.

266. The probiotic composition of any one of embodiments 1 to 261, wherein the probiotic composition maintains at least 60% of its CFU after 9 months of storage at 20° C.

267. The probiotic composition of any one of embodiments 1 to 261, wherein the probiotic composition maintains at least 80% of its CFU after 9 months of storage at 20° C.

268. The probiotic composition of any one of embodiments 1 to 261, wherein the probiotic composition maintains at least 60% of its CFU after 12 months of storage at 20° C.

269. The probiotic composition of any one of embodiments 1 to 261, wherein the probiotic composition maintains at least 80% of its CFU after 12 months of storage at 20° C.

270. The probiotic composition of any one of embodiments 1 to 261, wherein the probiotic composition maintains at least 60% of its CFU after 24 months of storage at 20° C.

271. The probiotic composition of any one of embodiments 1 to 261, wherein the probiotic composition maintains at least 80% of its CFU after 24 months of storage at 20° C.

272. The probiotic composition of any one of embodiments 1 to 271, wherein the non-human animal is a domesticated mammal.

273. The probiotic composition of any one of embodiments 1 to 272, wherein the animal is a ruminant.

274. The probiotic composition of embodiment 273, wherein the ruminant is a cow.

275. The probiotic composition of embodiment 274, wherein the cow is a dairy cow.

276. The probiotic composition of embodiment 274 or embodiment 275, wherein the cow is a breed of Bos taurus.

277. The probiotic composition of embodiment 276, wherein the breed is Holstein, Brown Swiss, Guernsey, Ayrshire, Jersey, Red and White, or Milking Shorthorn.

278. The probiotic composition of embodiment 277, wherein the breed is Holstein.

279. The probiotic composition of embodiment 274 or embodiment 275, wherein the cow is a breed of Bos indicus.

280. The probiotic composition of embodiment 279, wherein the breed is Sahiwal or Gir.

281. The probiotic composition of any one of embodiments 274 to 280, wherein the cow is a heifer.

282. The probiotic composition of any one of embodiments 274 to 280, wherein the cow is not a heifer.

283. The probiotic composition of embodiment 272, wherein the animal is a pig.

284. The probiotic composition of embodiment 272, wherein the animal is a horse.

285. A ready to use probiotic product comprising the probiotic composition of any one of embodiments 1 to 244, 249 to 252, or 256 to 284, except when depending from any of embodiments 245 to 248 or 253 to 255, in the form of a suppository.

286. A ready to use probiotic product comprising the probiotic composition of any one of embodiments 1 to 284 packaged within a capsule.

287. The probiotic product of embodiment 286, wherein the capsule is a gelatin capsule.

288. The probiotic product of embodiment 286, wherein the capsule is a pullulan capsule.

289. A ready to use probiotic product comprising the probiotic composition of any one of embodiments 1 to 284 packaged within a container.

290. The probiotic product of embodiment 289, wherein the container comprises a cartridge.

291. The probiotic product of embodiment 290, wherein the cartridge comprises 10 ml to 1000 ml of the probiotic composition.

292. The probiotic product of embodiment 291, wherein the cartridge comprises 10 to 100 ml of the probiotic composition.

293. The probiotic product of embodiment 291, wherein the cartridge comprises 100 to 200 ml of the probiotic composition.

294. The probiotic product of embodiment 291, wherein the cartridge comprises 200 to 1000 ml of the probiotic composition.

295. The probiotic product of embodiment 291, wherein the cartridge comprises 200 to 500 ml of the probiotic composition.

296. The probiotic product of embodiment 291, wherein the cartridge comprises 200 to 400 ml of the probiotic composition.

297. The probiotic product of embodiment 291, wherein the cartridge comprises 400 to 600 ml of the probiotic composition.

298. The probiotic product of embodiment 291, wherein the cartridge comprises 500 to 800 ml of the probiotic composition.

299. The probiotic product of embodiment 291, wherein the cartridge comprises 500 to 1000 ml of the probiotic composition.

300. The probiotic product of embodiment 291, wherein the cartridge comprises 300 ml of the probiotic composition.

301. The probiotic product of any one of embodiments 290 to 300, wherein the cartridge comprises a nozzle.

302. The probiotic product of any one of embodiments 290 to 301, wherein the cartridge comprises a removable cap.

303. The probiotic product of embodiment 289, wherein the container comprises a syringe.

304. The probiotic product of embodiment 303, wherein the syringe is a metered syringe.

305. The probiotic product of embodiment 303 or embodiment 304, wherein the syringe comprises 50 ml to 100 ml of the probiotic composition.

306. The probiotic product of embodiment 305, wherein the syringe comprises 60 ml or 80 ml of the probiotic composition.

307. The probiotic product of any one of embodiments 303 to 306, wherein the syringe comprises a removable cap.

308. The probiotic product of any one of embodiments 290 to 300, wherein the cartridge comprises an integrated applicator tube fixed to an end of the cartridge.

309. The probiotic product of embodiment 308, wherein the total length of the applicator tube is 6 to 18 inches.

310. The probiotic product of embodiment 309, wherein the length of the applicator tube is 6 to 15 inches.

311. The probiotic product of embodiment 309, wherein the length of the applicator tube is 6 to 12 inches.

312. The probiotic product of embodiment 309, wherein the length of the applicator tube is 6 to 9 inches.

313. The probiotic product of embodiment 309, wherein the length of the applicator tube is 9 to 18 inches.

314. The probiotic product of embodiment 309, wherein the length of the applicator tube is 9 to 15 inches.

315. The probiotic product of embodiment 309, wherein the length of the applicator tube is 9 to 12 inches.

316. The probiotic product of embodiment 309, wherein the length of the applicator tube is 12 to 18 inches.

317. The probiotic product of embodiment 309, wherein the length of the applicator tube is 12 to 15 inches.

318. The probiotic product of any one of embodiments 308 to 317, wherein the distal end of the applicator tube has an inner diameter which is ⅛ inches to ½ inches.

319. The probiotic product of embodiment 318, wherein the inner diameter of the distal end of the applicator tube is ¼ inches to ⅜ inches.

320. The probiotic product of embodiment 318, wherein the inner diameter of the distal end of the applicator tube is ¼ inches or ⅜ inches.

321. The probiotic product of any one of embodiments 308 to 320, wherein the cartridge comprises a removable cap.

322. A kit, comprising the probiotic product of any one of embodiments 289 to 307 and an applicator tube having a proximal end and a distal end, wherein the proximal end of the applicator tube is dimensioned to attach to an end of the container.

323. The kit of embodiment 322, wherein the inner diameter of the proximal end of the applicator tube is greater than the inner diameter of the distal end of the applicator tube.

324. The kit of embodiment 323, wherein the inner diameter of the proximal end of the applicator tube is ¼ to ¾ inches.

325. The kit of embodiment 324, wherein the inner diameter of the proximal end of the applicator tube is ½ inches.

326. The kit of any one of embodiments 323 to 325, wherein the inner diameter of the distal end of the applicator tube is ⅛ inches to ½ inches.

327. The kit of embodiment 326, wherein the inner diameter of the distal end of the applicator tube is ¼ inches to ⅜ inches.

328. The kit of embodiment 327, wherein the inner diameter of the distal end of the applicator tube is ¼ inches or ⅜ inches.

329. The kit of any one of embodiments 323 to 328, wherein the inner diameter of the applicator tube decreases uniformly from the proximal end to the distal end.

330. The kit of any one of embodiments 323 to 328, wherein the inner diameter of the applicator tube decreases non-uniformly from the proximal end to the distal end.

331. The kit of embodiment 330, wherein the inner diameter of the applicator tube decreases from the proximal end to the distal end by one or more step downs from a larger inner diameter to a smaller inner diameter.

332. The kit of embodiment 331, wherein the applicator tube comprises one step down.

333. The kit of embodiment 331, which comprises two step downs.

334. The kit of any one of embodiments 322 to 333, wherein the proximal end of the applicator tube comprises a flexible material.

335. The kit of embodiment 334, wherein the length of the flexible material is 3 to 5 inches long.

336. The kit of embodiment 335, wherein the length of the flexible material is 3 inches.

337. The kit of any one of embodiments 334 to 336, wherein the proximal end of the applicator tube comprises vinyl tubing.

338. The kit of any one of embodiments 322 to 337, wherein the distal end of the applicator tube comprises a rigid material.

339. The kit of embodiment 338, wherein the length of the rigid material is 5 to 13 inches.

340. The kit of embodiment 339, wherein the length of the rigid material is 7 to 11 inches.

341. The kit of embodiment 340, wherein the length of the rigid material is 9 inches.

342. The kit of any one of embodiments embodiment 338 to 341, wherein the rigid material comprises an acrylic rod or polycarbonate rod.

343. The kit of embodiment 342, wherein the rigid material comprises an acrylic rod.

344. The kit of embodiment 342, wherein the rigid material comprises a polycarbonate rod.

345. The kit of any one of embodiments 322 to 344, wherein the total length of the applicator tube is 6 to 18 inches.

346. The kit of embodiment 345, wherein the length of the applicator tube is 6 to 15 inches.

347. The kit of embodiment 345, wherein the length of the applicator tube is 6 to 12 inches.

348. The kit of embodiment 345, wherein the length of the applicator tube is 6 to 9 inches.

349. The kit of embodiment 345, wherein the length of the applicator tube is 9 to 18 inches.

350. The kit of embodiment 345, wherein the length of the applicator tube is 9 to 15 inches.

351. The kit of embodiment 345, wherein the length of the applicator tube is 9 to 12 inches.

352. The kit of embodiment 345, wherein the length of the applicator tube is 12 to 18 inches.

353. The kit of embodiment 345, wherein the length of the applicator tube is 12 to 15 inches.

354. The kit of embodiment 322, wherein the proximal end of the applicator tube comprises ¾ inch outer diameter (OD)×½ inch inner diameter (ID) tubing.

355. The kit of embodiment 354, wherein the distal end of the applicator tube comprises a rigid rod.

356. The kit of embodiment 355, wherein the applicator tube comprises:
(a) a 3 to 5 inch, optionally 3 inch, length of ¾ inch OD×½ inch ID tubing at the proximal end; and
(b) a 5 to 13 inch, optionally 9 inch, length ½ inch OD×⅜ inch ID rod at the distal end;

wherein the rod is positioned and fixed in an end of the tubing.

357. The kit of embodiment 355, wherein the applicator tube comprises:
(a) a 3 to 5 inch, optionally 3 inch, length of ¾ inch OD×½ inch ID tubing at the proximal end;
(b) a 1 inch to 4 inch, optionally 1.5 inch, length of ½ inch OD×⅜ inch ID tubing between the proximal and distal ends; and
(c) a 5 to 13 inch, optionally 9 inch, length ⅜ inch OD×¼ inch ID rod at the distal end;

wherein one end of the ½ inch OD×⅜ inch ID tubing is positioned and fixed in an end of the ¾ inch OD×½ inch ID tubing, and the rod is positioned and fixed in the other end of the ½ inch OD×⅜ inch ID tubing.

358. The kit of embodiment 355, wherein the applicator tube comprises:
(a) a 3 to 5 inch, optionally 3 inch, length of ¾ inch OD×½ inch ID tubing at the proximal end;
(b) a 5 to 13 inch, optionally 9 inch, length ½ inch OD×⅜ inch ID rod; and
(c) a ½ inch to 9 inch, optionally 1.5 inch, ⅜ inch OD×¼ inch ID rod;

wherein the ½ inch OD×⅜ inch ID rod is positioned and fixed in an end of the tubing, and the ⅜ inch OD×¼ inch ID rod is positioned and fixed in the distal end of the ½ inch OD×⅜ inch rod.

359. The kit of any one of embodiments 354 to 358, wherein the tubing is vinyl tubing.

360. The kit of any one of embodiments 354 to 359, wherein the rod is an acrylic rod or a polycarbonate rod.

361. The kit of embodiment 322, wherein the applicator tube is of uniform inner diameter over its length.

362. The kit of any one of embodiments 322 to 361, wherein the distal end of the applicator tube is burnished.

363. The kit of any one of embodiments 322 to 362, wherein the applicator tube is translucent along some or all of its length.

364. The kit of any one of embodiments 322 to 363, wherein the applicator tube further comprises an integrated clip for securing the applicator tube to the container.

365. The kit of any one of embodiments 322 to 363, wherein the kit further comprises a clip for securing the applicator tube to the container.

366. The kit of embodiment 364 or embodiment 365, wherein the clip comprises a hose clamp.

367. The kit of any one of embodiments 322 to 366, further comprising an applicator gun.

368. A kit comprising the probiotic product of any one of embodiments 308 to 321 and an applicator gun.

369. The kit of embodiment 367 or embodiment 368, wherein the applicator gun is configured to dispense a fixed volume of the probiotic composition with each trigger pull.

370. The kit of embodiment 369, wherein the fixed volume is in the range of 5 ml to 50 ml.

371. The kit of embodiment 369, wherein the fixed volume is in the range of 5 ml to 40 ml.

372. The kit of embodiment 369, wherein the fixed volume is in the range of 5 ml to 30 ml.

373. The kit of embodiment 369, wherein the fixed volume is in the range of 5 ml to 20 ml.

374. The kit of embodiment 369, wherein the fixed volume is in the range of 10 ml to 50 ml.

375. The kit of embodiment 369, wherein the fixed volume is in the range of 10 ml to 40 ml.

376. The kit of embodiment 369, wherein the fixed volume is in the range of 10 ml to 30 ml.

377. The kit of embodiment 369, wherein the fixed volume is in the range of 10 ml to 20 ml.

378. The kit of embodiment 369, wherein the fixed volume is in the range of 20 ml to 50 ml.

379. The kit of embodiment 369, wherein the fixed volume is in the range of 20 ml to 40 ml.

380. The kit of embodiment 369, wherein the fixed volume is in the range of 20 ml to 30 ml.

381. The kit of embodiment 369, wherein the fixed volume is in the range of 30 ml to 50 ml.

382. The kit of embodiment 369, wherein the fixed volume is in the range of 30 ml to 40 ml.

383. The kit of embodiment 369, wherein the fixed volume is in the range of 40 ml to 50 ml.

384. The kit of embodiment 369, wherein the fixed volume is in the range of 5 ml to 10 ml.

385. The kit of embodiment 369, wherein the fixed volume is 5 ml.

386. The kit of embodiment 369, wherein the fixed volume is 10 ml.

387. A system comprising the kit of any one of embodiments 322 to 386 in which the applicator tube is attached to the container.

388. A system comprising the kit of any one of embodiments 367 to 386, when depending from any one of embodiments 322 to 363, in which the applicator tube is attached to the container and the container is positioned in the applicator gun.

389. A system comprising the kit of any one of embodiments 368 to 386, when depending from any one of embodiments 308 to 321, in which the container is positioned in the applicator gun.

390. A method of introducing one or more strains of bacteria to the vagina of a non-human animal, comprising administering an amount of a probiotic composition to the vagina of the animal, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

391. The method of embodiment 390, wherein the probiotic composition is administered to the animal after a period of stress.

392. The method of embodiment 390, wherein the probiotic composition is administered to the animal before a period of stress.

393. The method of embodiment 390, wherein the probiotic composition is administered to the animal during a period of stress.

394. The method of any one of embodiments 390 to 393, wherein the probiotic composition is administered to the animal after antimicrobial therapy.

395. The method of any one of embodiments 390 to 393, wherein the probiotic composition is administered to the animal before antimicrobial therapy.

396. The method of any one of embodiments 390 to 393, wherein the probiotic composition is administered to the animal during antimicrobial therapy.

397. The method of any one of embodiments 394 to 396, wherein the antimicrobial therapy comprises an orally or intravenously administered antibiotic.

398. The method of embodiment 397, wherein the antibiotic is an antibiotic that was administered orally.

399. The method of embodiment 397, wherein the antibiotic is an antibiotic that was administered by injection.

400. A method of treating a uterine infection or lowering the risk of contracting a uterine infection in a non-human animal, comprising administering a therapeutically effective amount of a probiotic composition to the vagina of the animal, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

401. The method of embodiment 400, wherein the uterine infection comprises metritis.

402. The method of embodiment 400, wherein the uterine infection comprises endometritis.

403. The method of embodiment 400, wherein the uterine infection comprises pyometra.

404. The method of any one of embodiments 400 to 403, wherein the uterine infection comprises a bacterial infection, a viral infection, or a yeast infection.

405. The method of embodiment 404, wherein the uterine infection comprises a bacterial infection.

406. The method of embodiment 405, wherein the bacterial infection comprises an infection by one or more of Escherichia coli, Truperella pyogenes, Fusobacterium necrophorum, and Bacteroides melaminogenicus.

407. The method of embodiment 404, wherein the uterine infection comprises a viral infection.

408. The method of embodiment 404, wherein the uterine infection comprises a yeast infection.

409. A method of treating a urogenital infection or lowering the risk of contracting a urogenital infection in a female non-human animal, comprising administering a therapeutically effective amount of a probiotic composition to the vagina of the animal, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

410. The method of embodiment 409, wherein the urogenital infection comprises a bacterial infection, a viral infection, or a yeast infection.

411. The method of embodiment 410, wherein the urogenital infection comprises a bacterial infection.

412. The method of embodiment 411, wherein the bacterial infection comprises an infection by one or more of Escherichia coli, Truperella pyogenes, Fusobacterium necrophorum, and Bacteroides melaminogenicus.

413. The method of embodiment 410, wherein the urogenital infection comprises a viral infection.

414. The method of embodiment 410, wherein the urogenital infection comprises a yeast infection.

415. A method of promoting the establishment or maintenance of a heathy vaginal microbiome in non-human animal, comprising administering a therapeutically effective amount of a probiotic composition to the vagina of the animal, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

416. The method of embodiment 415, wherein the probiotic composition is administered to the animal after antimicrobial therapy.

417. The method of embodiment 416, wherein the antimicrobial therapy comprises an orally or intravenously administered antibiotic.

418. The method of embodiment 417, wherein the antibiotic is an antibiotic that was administered orally.

419. The method of embodiment 417, wherein the antibiotic is an antibiotic that was administered by injection.

420. The method of any one of embodiments 415 to 419, wherein the last administration of the antimicrobial therapy was less than one month before the first administration of the probiotic composition.

421. The method of embodiment 420, wherein the last administration of the antimicrobial therapy was less than 4 weeks before the first administration of the probiotic composition.

422. The method of embodiment 420, wherein the last administration of the antimicrobial therapy was less than 3 weeks before the first administration of the probiotic composition.

423. The method of embodiment 420, wherein the last administration of the antimicrobial therapy was less than 2 weeks before the first administration of the probiotic composition.

424. The method of embodiment 420, wherein the last administration of the antimicrobial therapy was less than 1 week before the first administration of the probiotic composition.

425. The method of any one of embodiments 390 to 424, wherein the amount of the probiotic composition administered contains 10⁸ to 10¹³ total CFU of bacteria.

426. The method of embodiment 425, wherein the amount of the probiotic composition administered contains 10⁸ to 10¹² total CFU of bacteria.

427. The method of embodiment 425, wherein the amount of the probiotic composition administered contains 10⁸ to 10¹¹ total CFU of bacteria.

428. The method of embodiment 425, wherein the amount of the probiotic composition administered contains 10⁸ to 10¹⁰ total CFU of bacteria.

429. The method of embodiment 425, wherein the amount of the probiotic composition administered contains 10⁸ to 10⁹ total CFU of bacteria.

430. The method of embodiment 425, wherein the amount of the probiotic composition administered contains 10⁹ to 10¹³ total CFU of bacteria.

431. The method of embodiment 425, wherein the amount of the probiotic composition administered contains 10⁹ to 10¹² total CFU of bacteria.

432. The method of embodiment 425, wherein the amount of the probiotic composition administered contains 10⁹ to 10¹¹ total CFU of bacteria.

433. The method of embodiment 425, wherein the amount of the probiotic composition administered contains 10⁹ to 10¹⁰ total CFU of bacteria.

434. The method of any one of embodiments 430 to 433, wherein the amount of the probiotic composition administered contains at least 4.5 billion total CFU of bacteria.

435. The method of embodiment 425, wherein the amount of the probiotic composition administered contains 10¹⁰ to 10¹² total CFU of bacteria.

436. The method of embodiment 425, wherein the amount of the probiotic composition administered contains 10¹⁰ to 10¹¹ total CFU of bacteria.

437. The method of embodiment 425, wherein the amount of the probiotic composition administered contains 10¹¹ to 10¹³ total CFU of bacteria.

438. The method of embodiment 425, wherein the amount of the probiotic composition administered contains 10¹¹ to 10¹² total CFU of bacteria.

439. The method of embodiment 425, wherein the amount of the probiotic composition administered contains 10¹² to 10¹³ total CFU of bacteria.

440. The method of any one of embodiments 390 to 439, which further comprises repeating the administration at least once.

441. The method of any one of embodiments 390 to 439, which further comprises repeating the administration at least twice.

442. The method of any one of embodiments 390 to 439, which further comprises repeating the administration at least three times.

443. The method of any one of embodiments 440 to 442, wherein each administration is separated from the subsequent administration by about 1 day to about 4 weeks.

444. The method of embodiment 443, wherein each administration is separated from the subsequent administration by about 1 day to about 1 week.

445. The method of embodiment 443, wherein each administration is separated from the subsequent administration by about 3 days to about 1 week.

446. The method of embodiment 443, wherein each administration is separated from the subsequent administration by about 1 week to about 3 weeks.

447. The method of embodiment 443, wherein each administration is separated from the subsequent administration by about 1 week to about 2 weeks.

448. The method of embodiment 443, wherein each administration is separated from the subsequent administration by about 2 weeks to about 4 weeks.

449. The method of embodiment 443, wherein each administration is separated from the subsequent administration by about 2 weeks to about 3 weeks.

450. The method of embodiment 443, wherein each administration is separated from the subsequent administration by about 3 weeks to about 4 weeks.

451. The method of embodiment 443, wherein each administration is separated from the subsequent administration by about 1 day.

452. The method of embodiment 443, wherein each administration is separated from the subsequent administration by about 2 days.

453. The method of embodiment 443, wherein each administration is separated from the subsequent administration by about 3 days.

454. The method of embodiment 443, wherein each administration is separated from the subsequent administration by about 4 days.

455. The method of embodiment 443, wherein each administration is separated from the subsequent administration by about 5 days.

456. The method of embodiment 443, wherein each administration is separated from the subsequent administration by about 6 days.

457. The method of embodiment 443, wherein each administration is separated from the subsequent administration by about 1 week.

458. The method of embodiment 443, wherein each administration is separated from the subsequent administration by about 2 weeks.

459. The method of embodiment 443, wherein each administration is separated from the subsequent administration by about 3 weeks.

460. The method of embodiment 443, wherein each administration is separated from the subsequent administration by about 4 weeks.

461. The method of any one of embodiments 390 to 439, wherein the animal is pregnant when the probiotic composition is administered for the first time or has given birth less than one month before the probiotic composition is administered for the first time.

462. The method of embodiment 461, wherein the animal is pregnant when the probiotic composition is administered for the first time.

463. A method of accelerating uterine involution in a non-human animal following labor, comprising administering a therapeutically effective amount of a probiotic composition to the vagina of the animal before and/or after labor, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

464. A method of accelerating resumption of ovarian cyclicity in a non-human animal following labor, comprising administering a therapeutically effective amount of a probiotic composition to the vagina of the animal before and/or after labor, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

465. A method of reducing the number of days open in a non-human animal following labor, comprising administering a therapeutically effective amount of a probiotic composition to the vagina of the animal before and/or after labor, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

466. A method of reducing the incidence of retained placenta in a non-human animal following labor, comprising administering a therapeutically effective amount of a probiotic composition to the vagina of the animal before and/or after labor, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

467. The method of any one of embodiments 461 to 466, which comprises administering the probiotic composition one or more times pre-partum.

468. The method of any one of embodiments 461 to 466, which comprises administering the probiotic composition at least two times pre-partum.

469. The method of embodiment 468, which comprises administering the probiotic twice pre-partum.

470. The method of any one of embodiments 461 to 469, which comprises administering the probiotic composition about 4 to 6 weeks prior to the expected date of labor.

471. The method of any one of embodiments 461 to 470, which comprises administering the probiotic composition about 1 to 2 weeks prior to the expected date of labor.

472. The method of any one of embodiments 461 to 471, which comprises administering the probiotic composition about 1 week prior to the expected date of labor.

473. The method of any one of embodiments 461 to 472, which comprises administering the probiotic composition about 2 weeks prior to the expected date of labor.

474. The method of embodiment 461, wherein the has given birth less than one month before the probiotic composition is administered for the first time.

475. The method of any one of embodiments 461 to 474, which comprises administering the probiotic composition one or more times post-partum.

476. The method of embodiment 475, which comprises administering the probiotic composition at least two times post-partum.

477. The method of embodiment 476, which comprises administering the probiotic twice post-partum.

478. The method of embodiment 476, which comprises administering the probiotic three times post-partum.

479. The method of embodiment 476, which comprises administering the probiotic four times post-partum.

480. The method of any one of embodiments 475 to 479, wherein each post-partum administration is separated from the subsequent administration by about 1 day.

481. The method of any one of embodiments 475 to 479, wherein each post-partum administration is separated from the subsequent administration by about 2 days.

482. The method of any one of embodiments 475 to 479, wherein each post-partum administration is separated from the subsequent administration by about 3 days.

483. The method of any one of embodiments 475 to 479, wherein each post-partum administration is separated from the subsequent administration by about 4 days.

484. The method of any one of embodiments 475 to 479, wherein each post-partum administration is separated from the subsequent administration by about 5 days.

485. The method of any one of embodiments 475 to 479, wherein each post-partum administration is separated from the subsequent administration by about 6 days.

486. The method of any one of embodiments 475 to 479, wherein each post-partum administration is separated from the subsequent administration by about 1 week.

487. The method of any one of embodiments 475 to 486, which comprises administering the probiotic composition within about one 1 week of labor.

488. The method of any one of embodiments 475 to 487, which comprises administering the probiotic composition about 1 week post-partum.

489. The method of any one of embodiments 475 to 488, which comprises administering the probiotic composition about 2 weeks post-partum.

490. A method of increasing an amount of colostrum and/or increasing the immunoglobulin content of colostrum produced by a non-human animal, comprising administering an amount of a probiotic composition of the disclosure to the vagina of a pregnant non-human animal prior to labor, wherein the probiotic composition is (a) a probiotic composition according to any one of embodiments 1 to 284, (b) a probiotic composition of a probiotic product according to any one of embodiments 285 to 321, (c) a probiotic composition of a kit according to any one of embodiments 322 to 386, or (d) a probiotic composition of a system according to any one of embodiments 387 to 389.

491. The method of embodiment 490, which comprises administering the probiotic composition one or more times prior to labor.

492. The method of embodiment 490, which comprises administering the probiotic composition at least two times prior to labor.

493. The method of embodiment 492, which comprises administering the probiotic twice prior to labor.

494. The method of any one of embodiments 490 to 493, which comprises administering the probiotic composition about 4 to 6 weeks prior to the expected date of labor.

495. The method of any one of embodiments 490 to 494, which comprises administering the probiotic composition about 1 to 2 weeks prior to the expected date of labor.

496. The method of any one of embodiments 490 to 495, which comprises administering the probiotic composition about 1 week prior to the expected date of labor.

497. The method of any one of embodiments 490 to 496, which comprises administering the probiotic composition about 2 weeks prior to the expected date of labor.

498. The method of any one of embodiments 390 to 497, wherein the non-human animal is a domesticated mammal.

499. The method of embodiment 498, wherein the animal is a ruminant.

500. The method of embodiment 499, wherein the ruminant is a cow.

501. The method of embodiment 500, wherein the cow is a dairy cow.

502. The method of embodiment 500 or embodiment 501, wherein the cow is a breed of Bos taurus.

503. The method of embodiment 502, wherein the breed is Holstein, Brown Swiss, Guernsey, Ayrshire, Jersey, Red and White, or Milking Shorthorn.

504. The method of embodiment 503, wherein the breed is Holstein.

505. The method of embodiment 500 or embodiment 501, wherein the cow is a breed of Bos indicus.

506. The method of embodiment 505, wherein the breed is Sahiwal or Gir.

507. The method of any one of embodiments 500 to 506, wherein the cow is a heifer.

508. The method of any one of embodiments 500 to 506, wherein the cow is not a heifer.

509. The method of embodiment 498, wherein the animal is a pig.

510. The method of embodiment 498, wherein the animal is a horse.

511. The method of any one of embodiments 390 to 510, wherein the probiotic composition is administered using an applicator.

512. The method of embodiment 511, wherein the applicator comprises a tube that is inserted into the vagina of the animal during administration.

513. The method of embodiment 512, when depending from any one of embodiments 500 to 508, wherein the applicator tube is inserted 3 inches to 12 inches into the vagina during administration.

514. The method of embodiment 513, wherein the applicator tube is inserted 6 to 9 inches into the vagina during administration.

515. The method of any one of embodiments 390 to 514, wherein the probiotic composition is a probiotic composition according to any one of embodiments 1 to 284.

516. The method of any one of embodiments 390 to 514, wherein the probiotic composition is a probiotic composition of a probiotic product according to any one of embodiments 289 to 321.

517. The method of any one of embodiments 390 to 514, wherein the probiotic composition is a probiotic composition of a kit according to any one of embodiments 322 to 386.

518. The method of embodiment 517, which further comprises, prior to administering the probiotic composition, assembling the components of the kit into a system for administering the probiotic composition.

519. The method of any one of embodiments 390 to 514, wherein the probiotic composition is administered using the system of any one of embodiments 387 to 389.

520. The method of embodiment 519, which further comprises assembling the system prior to administering the probiotic composition.

While various specific embodiments have been illustrated and described, it will be appreciated that various changes can be made without departing from the spirit and scope of the disclosure(s).

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A probiotic composition in gel form suitable for intravaginal administration to a non-human animal, comprising:
(a) one or more strains of bacteria native to the vaginal tracts of healthy animals; and
(b) a non-aqueous base.

2. The probiotic composition of claim 1, wherein the one or more strains of bacteria comprise one or more strains of lactic acid bacteria (LAB).

3. The probiotic composition of claim 1, wherein the one or more strains of bacteria are dried.

4. The probiotic composition of claim 1, wherein the non-aqueous base comprises one or more oils.

5. The probiotic composition of claim 4, wherein the non-aqueous base further comprises one or more thickeners.

6. The probiotic composition of claim 5, wherein the one or more thickeners comprise pullulan.

7. The probiotic composition of claim 4, wherein the non-aqueous base further comprises one or more prebiotics.

8. The probiotic composition of claim 7, wherein the one or more prebiotics comprise one or more fermentation products.

9. The probiotic composition of claim 1, wherein the probiotic composition has a specific gravity ranging from 1.0 to 1.2.

10. The probiotic composition of claim 1, wherein the probiotic composition is not runny over the temperature range of 10° C. to 70° C.

11. The probiotic composition of claim 1, wherein the probiotic composition maintains at least 60% of its CFU after 3 months of storage at 20° C.

12. A ready to use probiotic product comprising the probiotic composition of claim 1 packaged within a container.

13. A ready to use probiotic product comprising the probiotic composition of claim 1 in the form of a suppository.

14. A ready to use probiotic product comprising the probiotic composition of claim 1 packaged within a capsule.

15. A kit, comprising the probiotic product of claim 12 and an applicator tube having a proximal end and a distal end, wherein the proximal end of the applicator tube is dimensioned to attach to an end of the container.

16. A system comprising the kit of claim 15 in which the applicator tube is attached to the container.

17. A method of introducing one or more strains of bacteria to the vagina of a non-human animal, comprising administering an amount of the probiotic composition of claim 1 the vagina of the animal.

18. The method of claim 17, wherein the non-human animal is cow, horse, pig, goat, or sheep.

19. The method of claim 17, wherein the probiotic composition is administered to the animal before or during a period of stress.

20. The method of claim 17, wherein the probiotic composition is administered to the animal after a period of stress.

21. The method of claim 17, which further comprises repeating the administration at least once.

22. The method of claim 21, wherein each administration is separated from the subsequent administration by about 1 day to about 4 weeks.

23. The method of claim 17, wherein the animal is pregnant when the probiotic composition is administered for the first time or has given birth less than one month before the probiotic composition is administered for the first time.

24. The method of any claim 23, which comprises administering the probiotic composition one or more times pre-partum.

25. The method of claim 23, which comprises administering the probiotic composition about 4 to 6 weeks prior to the expected date of labor and about 1 to 2 weeks prior to the expected date of labor.

26. The method of claim 23, which comprises administering the probiotic composition about 2 weeks prior to the expected date of labor and about 1 week prior to the expected date of labor.

27. The method of claim 23, which comprises administering the probiotic composition one or more times post-partum.

28. The method of claim 27, which comprises administering the probiotic composition about 1 week post-partum and about 2 weeks post-partum.

29. The method of claim 23, which comprises administering the probiotic composition about 4 to 6 weeks prior to the expected date of labor, about 1 to 2 weeks prior to the expected date of labor, about 1 week post-partum and about 2 weeks post-partum.

30. A method of treating a uterine infection or lowering the risk of contracting a uterine infection in a non-human animal, comprising administering a therapeutically effective amount of the probiotic composition of claim 1 to the vagina of the animal.

31. A method of promoting the establishment or maintenance of a heathy vaginal microbiome in non-human animal, comprising administering a therapeutically effective amount of the probiotic composition of claim 1 to the vagina of the animal.
